(19) United States
(12) Patent Application Publication
(10) Pub. No.: US 2021/0237080 A1
(43) Pub. Date: Aug. 5, 2021

(54) SCREENING PLANT PROTOPLASTS FOR DISEASE RESISTANT TRAITS

(71) Applicant: Berkeley Lights, Inc., Emeryville, CA (US)

(72) Inventors: Troy A. LIONBERGER, Emeryville, CA (US); Volker L.S. KURZ, Emeryville, CA (US)

(21) Appl. No.: 17/140,861

(22) Filed: Jan. 4, 2021

Related U.S. Application Data
(63) Continuation of application No. PCT/US19/41692, filed on Jul. 12, 2019.
(60) Provisional application No. 62/697,199, filed on Jul. 12, 2018.

(51) Int. Cl. B01L 3/00; G01N 33/00

(52) U.S. Cl. CPC B01L 3/502761; B01L 3/502769; G01N 33/0098; B01L 2200/0668; B01L 2400/086; B01L 2300/0819; B01L 2300/0877; B01L 2400/0424; B01L 2400/0454; B01L 2300/0816

(57) ABSTRACT

Methods for screening plant cells, particularly plant protoplasts, for disease resistant traits, and kits for performing such methods are provided. The methods are performed in a microfluidic device that includes a flow region and at least one growth chamber suitable for culturing and screening a plant protoplast. The at least one surface of the growth chamber of the microfluidic chip can include a covalently linked coating material or a surface modifying ligand. The kit can comprise a microfluidic chip in combination with a reagent for detecting the viability of the plant protoplast and, optionally, a surface conditioning reagent or a surface modification reagent.

### CROSS-REFERENCE TO RELATED APPLICATION

This application claims the benefit under 35 U.S.C. § 119(e) of U.S. Provisional Application No. 62/697,199, filed Jul. 12, 2019, the contents of which are hereby incorporated by reference herein.

### BACKGROUND OF THE DISCLOSURE

In biosciences and related fields, it can be useful to culture cells, particularly single cells, under conditions that allow the cells to be monitored and/or assays so that cells of interest can be isolated for further study or use. Unfortunately, suitable culture conditions remain unknown or non-optimized for most types of cells. Some embodiments disclosed herein include processes for culturing plant protoplasts in a microfluidic device. The protoplasts can be cultured individually or in groups. Other embodiments disclosed herein include processes for screening plant protoplasts for desirable traits, such as disease resistance genes, while culturing the protoplasts in a microfluidic device.

### SUMMARY OF THE DISCLOSURE

In one aspect, a method of identifying a plant protoplast that lacks pathogen resistance is disclosed. The method can comprise: introducing a first fluidic medium containing one or more plant protoplasts into a microfluidic device comprising an enclosure having a flow region and at least one growth chamber; moving a first protoplast of the one or more protoplasts into a first growth chamber of the at least one growth chamber; contacting the first protoplast with a pathogenic agent; and monitoring viability of the first protoplast during a first time period after contacting the first protoplast with the pathogenic agent. Protoplast viability at the end of the first time period indicates that the protoplast lacks resistance to the pathogenic agent. Such protoplasts can be exported from their corresponding growth chambers and recovered off-chip for further analysis (e.g., sequencing to determine the molecular basis for the lack of pathogen resistance). In certain embodiments, the method further comprises moving at least one protoplast into each of a plurality of growth chambers in the microfluidic device and performing the remaining steps of the method on all of the protoplasts moved into the plurality of growth chambers.

In certain embodiments, the method is performed using protoplasts from: a broad acre crop plant, such as a wheat, corn, soy, or cotton plant; a high value or ornamental crop plant, such as a tomato, lettuce, pepper, or squash plant; a turf or forage plant, such as a grass or alfalfa plant; or an experimental plant, such as an Arabidopsis plant or an Antirrhinum plant.

In certain embodiments, the pathogenic agent is a plant pathogen or a molecule derived therefrom. The plant pathogen can be a virus, a bacterium, a fungal cell, or the like. In certain embodiments, the pathogenic agent is a molecular agent derived from the plant pathogen (e.g., a viral capsid protein, a flagellar protein, a lipopolysaccharide, a peptidoglycan, a chitin protein) or a fragment thereof.

In another aspect, a kit for performing a method of identifying a plant protoplast that lacks pathogen resistance is disclosed. The kit can include a microfluidic chip and a reagent for detecting the viability of the plant protoplast. The microfluidic chip can have a configuration according to any of the microfluidic chips disclosed herein. For example, the microfluidic chip can include an enclosure having a flow region and at least one growth chamber and, optionally, at least one surface of the growth chamber can include a surface modifying ligand or a covalently linked coating material. The reagent for detecting the viability of the plant protoplast can be a fluorescent stain, such as fluorescein diacetate (FDA), Hoechst, calcofluor white, a chlorophyll stain, or the like.

These and other features and advantages of the disclosed methods and kits will be set forth or will become more fully apparent in the description that follows and in the appended claims. The features and advantages may be realized and obtained by means of the objects and combinations particularly pointed out in the appended examples, partial listing of embodiments, and claims. Furthermore, the features and advantages of the described methods may be learned by the practice or will be obvious from the description, as set forth hereinafter.

### BRIEF DESCRIPTION OF THE DRAWINGS

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A-2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A-4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6 is an example of one embodiment of a process for perfusing a fluidic medium in a microfluidic device.

FIG. 7 is an example of another embodiment of a process for perfusing a fluidic medium in a microfluidic device.

FIG. 8 depict photographic representations of grape protoplasts cultured according to one embodiment of the methods described herein.

FIG. 9 depict photographic representations of lettuce protoplasts cultured according to one embodiment of the methods described herein.

FIG. 10 is a schematic diagram for a method of genotyping plant protoplasts according to the methods described herein.

FIG. 11 is a schematic diagram for a method of identifying disease-resistance traits according to the methods described herein.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to an x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include or be a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 microliters. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 microliters. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 microliters, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. No. 6,408,878 (Unger et al.) and U.S. Pat. No. 9,227,200 (Chiou et al.), each of which is herein incorporated by reference in its entirety.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result.

The phrase “substantially no flow” refers to a rate of flow of a fluidic medium that, averaged over time, is less than the rate of diffusion of components of a material (e.g., an analyte of interest) into or within the fluidic medium. The rate of diffusion of components of such a material can depend on, for example, temperature, the size of the components, and the strength of interactions between the components and the fluidic medium.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the microfluidic device.

As used herein, a “flow path” refers to one or more fluidically connected circuit elements (e.g. channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” constitutes confining a micro-object to a defined area within the microfluidic device.

As used herein, an “isolation region” refers to a region within a microfluidic device that is configured to hold a micro-object such that the micro-object is not drawn away from the region as a result of fluid flowing through the microfluidic device. Depending upon context, the term “isolation region” can further refer to the structures that define the region, which can include a base/substrate, walls (e.g., made from microfluidic circuit material), and a cover.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light which illuminates a portion of a surface of a device without illuminating an adjacent portion of the surface. Structured light is typically generated by a structured light modulator, such as a digital mirror device (DMD), a microshutter array system (MSA), a liquid crystal display (LCD), or the like. Structured light may be corrected for surface irregularities and for irregularities associated with the light projection itself, e.g., image fall-off at the edge of an illuminated field.

As used herein, the “clear aperture” of a lens (or lens assembly) is the diameter or size of the portion of the lens (or lens assembly) that can be used for its intended purpose. In some instances, the clear aperture can be substantially equal to the physical diameter of the lens (or lens assembly). However, owing to manufacturing constraints, it can be difficult to produce a clear aperture equal to the actual physical diameter of the lens (or lens assembly).

As used herein, the term “active area” refers to the portion of an image sensor or structured light modulator that can be used, respectively, to image or provide structured light to a field of view in a particular optical apparatus. The active area is subject to constraints of the optical apparatus, such as the aperture stop of the light path within the optical apparatus. Although the active area corresponds to a two-dimensional surface, the measurement of active area typically corresponds to the length of a diagonal line through opposing corners of a square having the same area.

As used herein, an “image light beam” is an electromagnetic wave that is reflected or emitted from a device surface, a micro-object, or a fluidic medium that is being viewed by an optical apparatus. The device can be any microfluidic device as described herein. The micro-object and the fluidic medium can be located within such a microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, nucleic acids (e.g., oligonuclewotides), proteins, carbohydrates, antigens, small molecule signaling moieties, or other chemical/biological species capable of use in an assay. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include: eukaryotic cells, plant cells, animal cells, such as mammalian cells, reptilian cells, avian cells, fish cells, or the like; prokaryotic cells, bacterial cells, fungal cells, protozoan cells, or the like; cells dissociated from a tissue, such as muscle, cartilage, fat, skin, liver, or lung cells, neurons, glial cells, and the like; immunological cells, such as T cells, B cells, plasma cells, natural killer cells, macrophages, and the like; embryos (e.g., zygotes), germ cells, such as oocytes, ova, and sperm cells, and the like; fusion cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a pig, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the terms “maintaining a cells” and “maintaining cells” refer to providing an environment comprising both fluidic and gaseous components and, optionally a surface, that provides the conditions necessary to keep the cell (s0 viable and/or expanding.

As used herein, the term “expanding” when referring to cells, refers to increasing in cell number.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein, “capture moiety” is a chemical or biological species, functionality, or motif that provides a recognition site for a micro-object. A selected class of micro-objects may recognize the in situ-generated capture moiety and may bind or have an affinity for the in situ-generated capture moiety. Non-limiting examples include antigens, antibodies, and cell surface binding motifs.

As used herein, “antibody” refers to an immunoglobulin (Ig) and includes both polyclonal and monoclonal antibodies; multichain antibodies, such as IgG, IgM, IgA, IgE, and IgD antibodies; single chain antibodies, such as camelid antibodies; mammalian antibodies, including primate antibodies (e.g., human), rodent antibodies (e.g., mouse, rat, guinea pig, hamster, and the like), lagomorph antibodies (e.g., rabbit), ungulate antibodies (e.g., cow, pig, horse, donkey, camel, and the like), and canidae antibodies (e.g., dog); primatized (e.g., humanized) antibodies; chimeric antibodies, such as mouse-human, mouse-primate antibodies, or the like; and may be an intact molecule or a fragment thereof (such as a light chain variable region (VL), heavy chain variable region (VH), scFv, Fv, Fd, Fab, Fab′ and F(ab)′2 fragments), or multimers or aggregates of intact molecules and/or fragments; and may occur in nature or be produced, e.g., by immunization, synthesis or genetic engineering. An “antibody fragment,” as used herein, refers to fragments, derived from or related to an antibody, which bind antigen. In some embodiments, antibody fragments may be derivatized to exhibit structural features that facilitate clearance and uptake, e.g., by the incorporation of galactose residues. The capability of biological micro-objects (e.g., biological cells) to produce specific biological materials (e.g., proteins, such as antibodies) can be assayed in such a microfluidic device. In a specific embodiment of an assay, sample material comprising biological micro-objects (e.g., cells) to be assayed for production of an analyte of interest can be loaded into a swept region of the microfluidic device. Ones of the biological micro-objects (e.g., mammalian cells, such as human cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

An antigen, as referred to herein, is a molecule or portion thereof that can bind with specificity to another molecule, such as an Ag-specific receptor. An antigen may be any portion of a molecule, such as a conformational epitope or a linear molecular fragment, and often can be recognized by highly variable antigen receptors (B-cell receptor or T-cell receptor) of the adaptive immune system. An antigen may include a peptide, polysaccharide, or lipid. An antigen may be characterized by its ability to bind to an antibody's variable Fab region. Different antibodies have the potential to discriminate among different epitopes present on the antigen surface, the structure of which may be modulated by the presence of a hapten, which may be a small molecule.

The capability of biological micro-objects (e.g., biological cells) to produce specific biological materials (e.g., proteins, such as antibodies) can be assayed in a microfluidic device. In a specific embodiment of an assay, sample material comprising biological micro-objects (e.g., cells) to be assayed for production of an analyte of interest can be loaded into a swept region of the microfluidic device. Ones of the biological micro-objects (e.g., plant cells, such as plant protoplasts) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

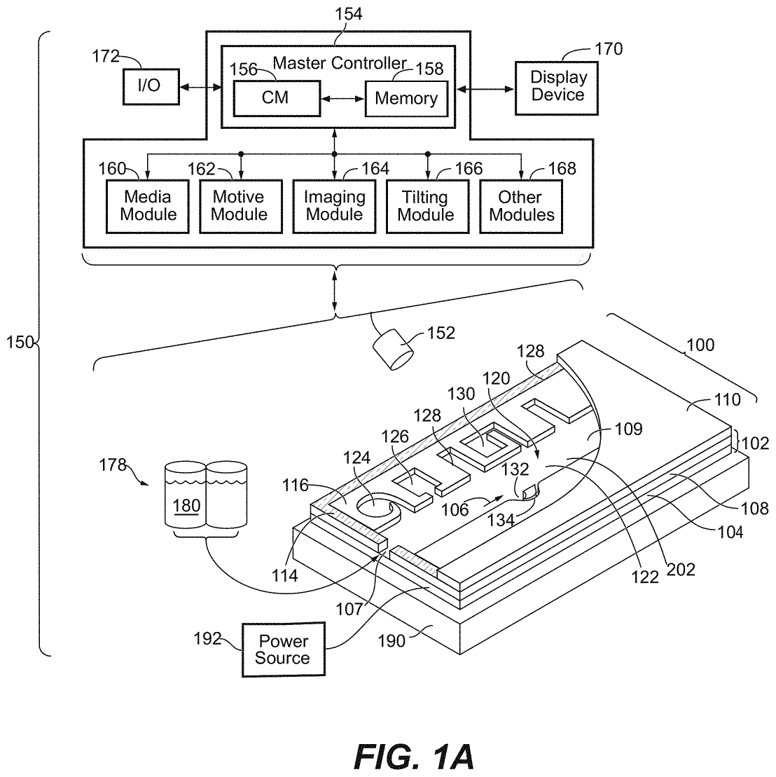

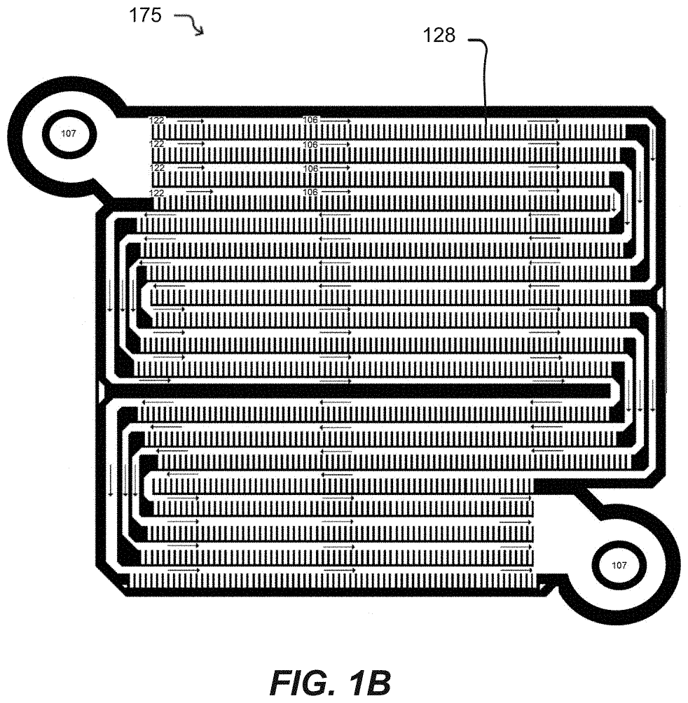

Microfluidic device/system feature cross-applicability. It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g. rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g. photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n−1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1n, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

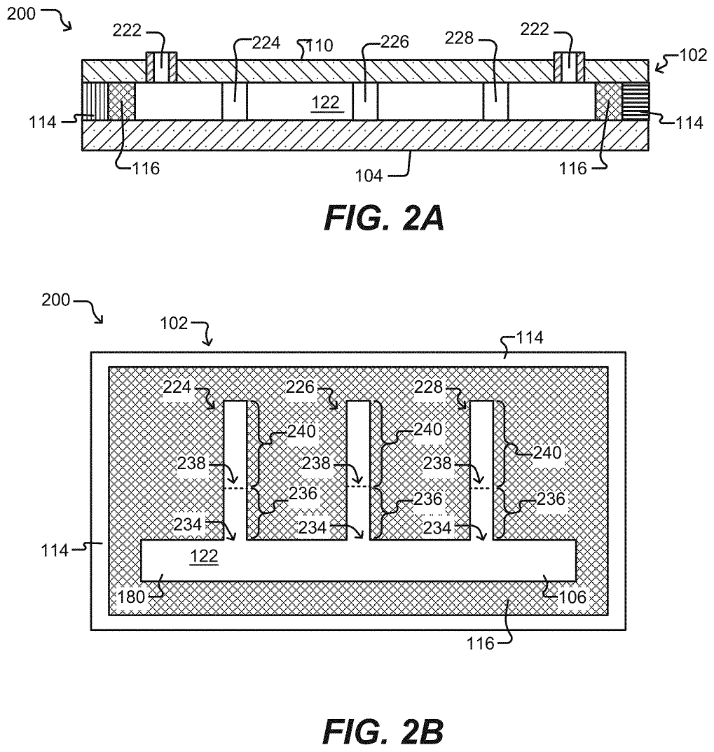

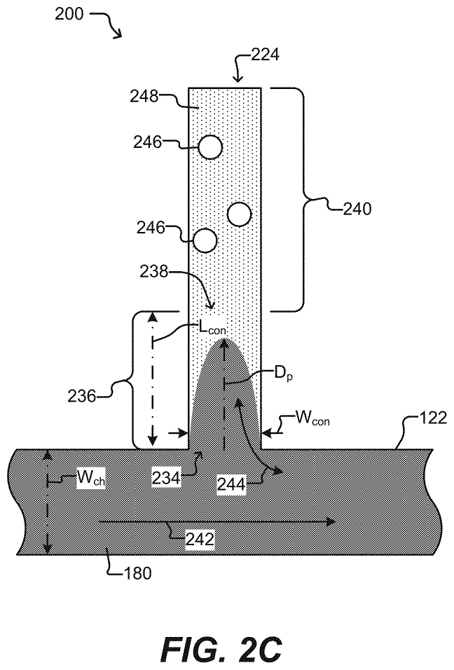

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%. 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180. In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g. a portion of the connection region adjacent to the proximal opening 234).

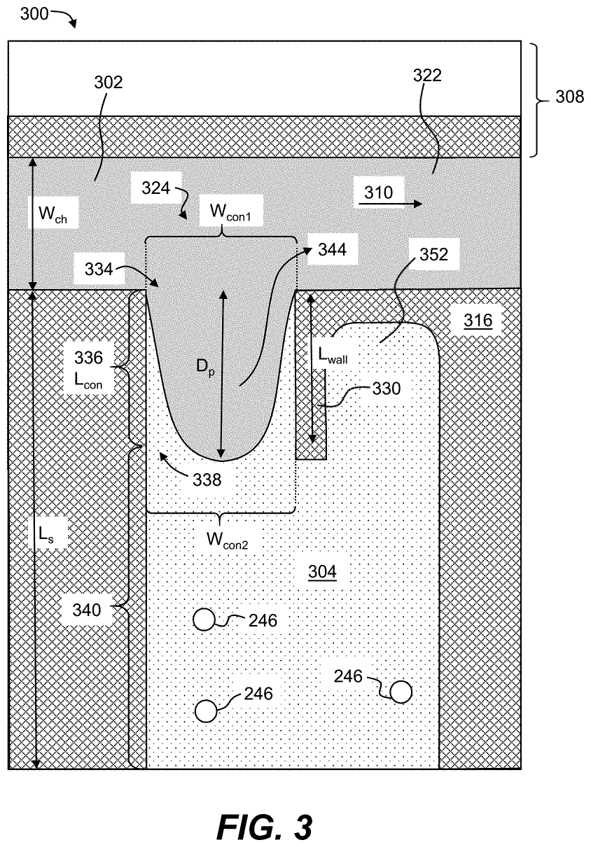

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 includes a microfluidic channel 322, having a width W_{ch}, as described herein, and containing a flow 310 of first fluidic medium 302 and one or more sequestration pens 324 (only one illustrated in FIG. 3). The sequestration pens 324 each have a length Ls, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Sequestration pen dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure, as follows.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25. times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Ls of about 40-600 microns, about 40-500 microns, about 40-400 microns, about 40-300 microns, about 40-200 microns, about 40-100 microns or about 40-80 microns. The foregoing are examples only, and a sequestration pen may have a length Ls selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height Heh of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, or 20 microliters/sec. The foregoing are examples only, and the flow of fluidic medium within the microfluidic channel can have a maximum velocity (e.g., Vₘₐₓ) selected to be a value between any of the values listed above.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules:second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g. vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

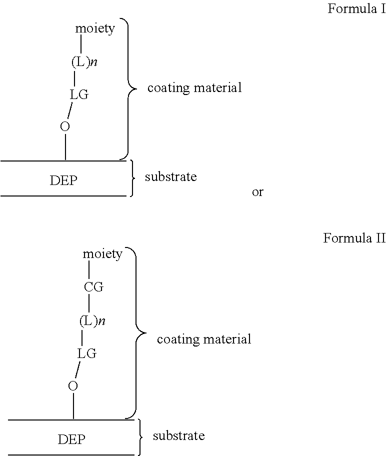

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Pat. No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

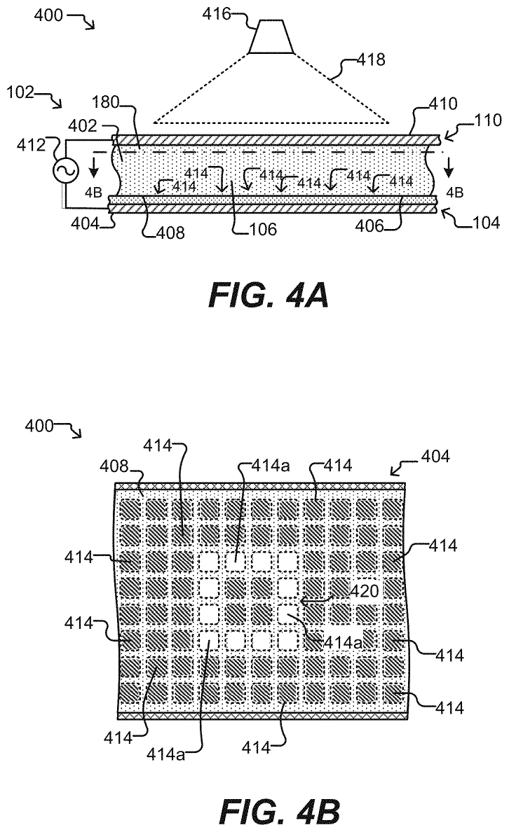

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 200 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 414 can be created anywhere and in any pattern on the inner surface 408 of the electrode activation substrate 406, in accordance with the light pattern 418. The number and pattern of the DEP electrode regions 214 thus need not be fixed, but can correspond to the light pattern 418. Examples of microfluidic devices having a DEP configuration comprising a photoconductive layer such as discussed above have been described, for example, in U.S. Pat. No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrates that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can be also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied at the same time as the other forces or in an alternating manner with the other forces.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g. for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g. a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e. at 0° relative to x- and y-axes), a vertical orientation (i.e. at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35° 40°, 45° 50°, 55° 60°, 65°, 70°, 75° 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3° 4° 5° or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e. an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

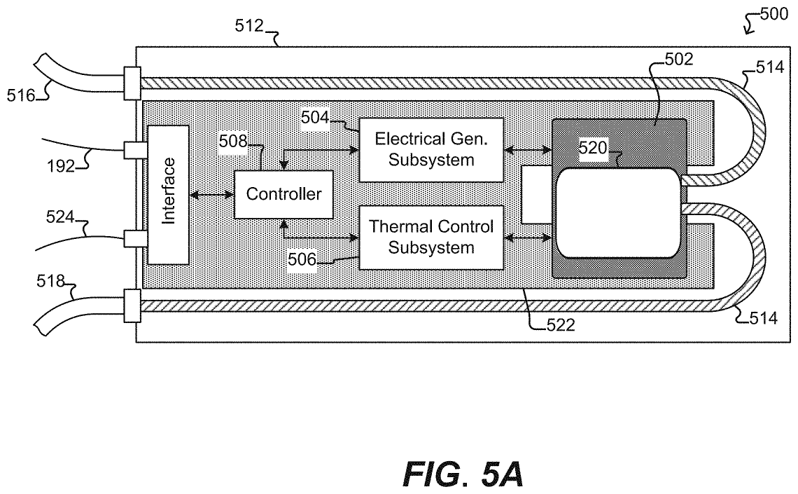

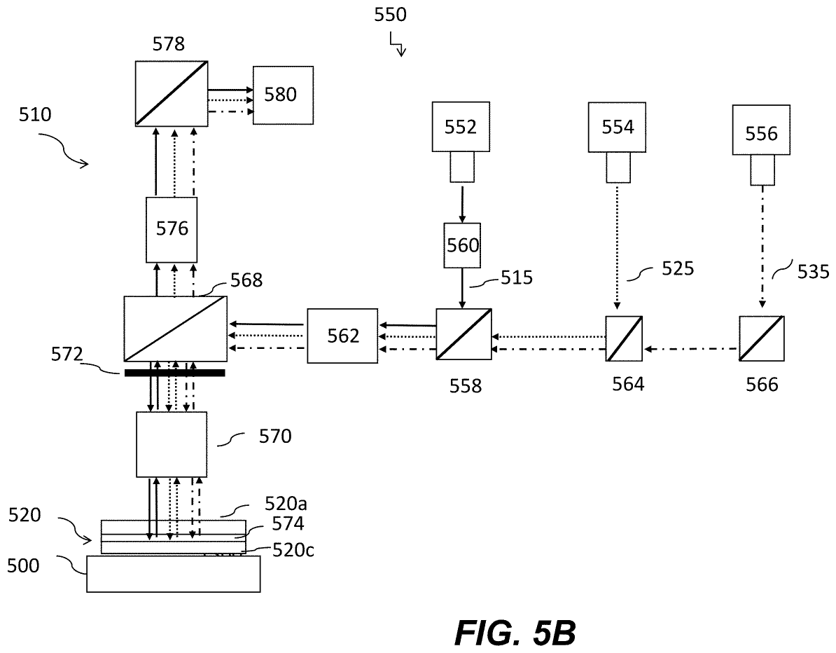

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically-actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 322, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 3A the controller 308 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns×10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 5621 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive light 535 from the third light source 556), and the second light, brightfield illumination 525, may be transmitted therefrom to the first dichroic beam splitter 558. The second light, brightfield illumination 525, may then be transmitted from the first beam splitter 558 to the first tube lens 562.

The third light source 556 can transmit light through a matched pair relay lens (not shown) to a mirror 566. The third light illumination 535 may therefrom be reflected to the second dichroic beam splitter 5338 and be transmitted therefrom to the first beam splitter 5338, and onward to the first tube lens 5381. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 5560) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 1350. The objective lens 570 can have one or more magnification levels available such as, 4×, 10×, 20×.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 560 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns×40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), U.S. Pat. No. 10,010,882 (White et al.), and U.S. Pat. No. 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Cells. A cell capable of use in the system and methods of the disclosure may be any type of plant protoplast. For example, the protoplast can be from any type of plant used for agriculture. Non-limiting examples of agricultural plants include: broad acre crop plants, such as a wheat, corn, soy, or cotton plant; high value crop plants, such as a tobacco, tomato, lettuce, pepper, or squash plant; a brassica plant, such as a broccoli, brown mustard, brussels sprouts, cabbage, cauliflower, kale, kohlrabi, rape, rutabaga, turnip, or Arabidopsis plant; an ornamental plant, such as a rose, petunia, poppy, lilly, lavender, silver grass, or cactus plant; a fruit tree, shrub, or vine, such as a grape, apple, orange, strawberry, blackberry, blueberry, raspberry, plum, pluot, apricot plant, or the like; or a turf or forage plant, such as a grass or alfalfa plant. Methods of obtaining protoplasts are known in the art and have been described in, for example: Giles, Kenneth, editor. Plant Protoplasts: International Review of Cytology, Vol. 16, Academic Press 1983; Yoo et al. (2007), Nature Protocols, Vol. 2(7), 1565-72; and Danon (2014), Bio-Protocol, Vol. 4(12), e1149.

In some embodiments, the cell may be from a population of cells actively growing in culture or obtained from a fresh tissue sample (e.g., by dissociation of a solid tissue sample, such as a plant leaf, stem, root, flower, etc.). Alternatively, the one or more biological cells can be from a culture of other sample that was previously frozen.

Depending on the particular goal of the experiment, only one cell or a plurality of cells may be introduced into the growth chamber (e.g., a sequestration pen) of the microfluidic device for culturing and/or cloning. When only one cell is introduced into a growth chamber of the system and incubated according to the methods described herein, the resulting expanded population is a clonal colony of the cell originally introduced into the growth chamber.

Methods. A method is provided for culturing and assaying at least one cell, particularly a plant protoplast, in a system including a microfluidic device having at least one growth chamber and a flow region. Culturing a cell (or cells) in a microfluidic growth chamber having a nanoliter-scale volume can facilitate the culturing of cells that otherwise can't be cultured. For example, a single cell in a 1 nanoliter volume chamber has an effective concentration of 1×10⁶ cells/mL. Because of the small volume of the chamber, proteins and other molecules released into culture can rapidly condition the medium in the chamber, ensuring that the cell receives signals necessary for supporting cell viability. In addition, culturing a cell (or cells) in a growth chamber of a microfluidic device having a flow region can allow specific introduction of nutrients, growth factors or other cell signaling species at selected periods of time to achieve control of cell growth, viability, or portability parameters. The precise control of cell placement/removal and of nutrient/signaling/environmental stimuli made possible by the methods described herein is difficult or impossible to achieve with macroscale culturing or other microfluidic culturing methods.

The at least one biological cell (e.g., plant protoplast) can be introduced into a growth chamber having at least one conditioned surface, where the conditioned surface supports cell growth, viability, portability, or any combination thereof, as discussed above. In some embodiments, the conditioned surface supports cell portability within the microfluidic device. In some embodiments, portability includes preventing non-specific adhesion of cells to the microfluidic device. The at least one conditioned surface may be any conditioned surface as described herein. The conditioned surface may be covalently linked to the microfluidic device. In some embodiments, the conditioned surface may include a linking group covalently linked to the surface, and the linking group may also be linked to a moiety configured to support cell growth, viability, portability, or any combination thereof, of the one or more biological cells within the microfluidic device. In some embodiments, a microfluidic device having a conditioned surface may be provided prior to importation of the one or more biological cells. The introduction of the biological cell may be accomplished using a number of different motive forces, as described herein, some of which may permit precise control in placing a specific biological cell into a specific location on the microfluidic device, for example, into a preselected growth chamber.

After placement, the at least one biological cell is then incubated for a period of time at least long enough to expand the at least one biological cell to produce a colony of biological cells. When biological cells (e.g., plant protoplasts) are introduced into separate growth chambers, the resulting expanded colonies can be precisely identified for further use as separable groups of biological cells. When only one biological cell is introduced to a growth chamber and allowed to expand, the resulting colony is a clonal population of biological cells. Any appropriate cell may be used in the methods, including but not limited to the cells as described above.

The microfluidic device may be any of microfluidic devices 100, 300, 400, 500A-E, or 600 as described herein, and the microfluidic device may be part of a system having any of the components as described herein. The at least one growth chamber may include a plurality of growth chambers, and any suitable number of growth chambers as discussed herein may be used.

Introducing at least one biological cell. In some embodiments, introducing the at least one biological cell (e.g., plant protoplast) into the at least one growth chamber may include using a dielectrophoresis (DEP) force having sufficient strength to move the at least one biological cell. The DEP force may be produced using electronic tweezers, such as optoelectronic tweezers (OET). In some other embodiments, introducing one or more biological cells into the at least one growth chamber may include using fluid flow and/or gravity (e.g., by tilting the microfluidic device such that the cell(s) drop into a growth chamber located beneath the cell(s).

In some embodiments, the at least one biological cell (e.g., plant protoplast) is introduced into the microfluidic device through an inlet port 124 into a flow region (e.g., flow channel) of the microfluidic device. The flow of medium in the flow channel can carry the cell to a location proximal to an opening to a growth chamber. After being position proximal to an opening to a growth chamber, the biological cell may then be moved into the growth chamber using any of the motive forces described herein, including dielectrophoresis or gravity. Dielectrophoresis forces can include electrically actuated or optically actuated forces, and the DEP forces may further be provided by optoelectronic tweezers (OET). The at least one biological cell may be moved through the flow channel to the proximal opening of a connection region of at least one growth chamber, where the connection region opens directly to and is fluidically connected to the flow channel/region. The connection region of the at least one growth chamber is also fluidically connected to an isolation region of the at least one growth chamber. The at least one biological cell may further be moved through the connection region and into the isolation region of the at least one growth chamber. The isolation region of the at least one growth chamber may have dimensions sufficient to support cell expansion. Typically, however the dimensions of the growth chamber will limit such expansion to no more than about 1×10², 50, 25, 15, or even as few as 10 cells in culture. In some embodiments, the isolation region may have dimensions sufficient to support cell expansion to no more than about 1×10², 50, 25, 15, or 10 cells in culture. It has been surprisingly found that protoplast incubation and/or expansion up to about 20 or more cells may be successfully performed in an isolation region having a volume of no more than 1.5×10⁶ cubic microns, or 1.0×10⁶ cubic microns. Depending on the protoplast type, the cell diameter may vary greatly. Accordingly, a growth chamber having a volume of about 5×10⁵ cubic microns may permit expansion of only a few protoplasts cells having a large diameter (e.g., about 30 microns to about 50 microns in diameter), whereas the same small growth chamber (volume of about 5×10⁵ cubic microns) may permit greater expansion of protoplasts having a smaller diameter (e.g., about 10 microns to about 30 microns in diameter).

The method may further include introducing a first fluidic medium into a microfluidic channel of the flow region of the microfluidic device. In some embodiments, introduction of the first fluidic medium is performed prior to introducing the at least one plant protoplast. When the first fluidic medium is introduced before introducing the at least one plant protoplast, a flow rate may be selected such that the first fluidic medium is flowed into the growth chamber from the flow channel of the microfluidic device, e.g. at any suitable rate. Alternatively, if the microfluidic device has been primed with a medium containing an excess of one or more conditioning reagents, the first fluidic medium is flowed into the microfluidic channel at a rate such that the first fluidic medium replaces any remaining medium containing excess conditioning reagent(s) in the flow region.

When the flow of the first fluidic medium is introduced after introduction of the at least one plant protoplast to the growth chamber, the flow rate of the first fluidic medium may be selected to not sweep the isolation region which will not displace the at least one plant protoplast from the isolation region. The fluidic medium surrounding the at least one plant protoplast in the isolation region of the at least one growth chamber is the second fluidic medium, which may be the same or different from the first fluidic medium. In some embodiments, the second fluidic medium may be the same as the first fluidic medium, but during the incubating step, cellular waste products and depleted medium components may render the second fluidic medium different from the first fluidic medium.

Incubating the cell. In the methods described herein, the at least one plant protoplast is incubated for a period of time at least long enough to expand the cell to produce a colony of biological cells. That period of time may be selected to be from about 1 day to about 14 days. In other embodiments, the incubation period may be extended beyond 14 days and may continue for any desired period. Since the cells in the isolation region of the growth chamber are provided with nutrients and have waste removed by perfusion of fluidic medium, cells may be grown indefinitely. As the isolation region fills with the expanded cell population, any additional expansion will result in expanded plant protoplasts inhabiting the connection region of the growth chamber, which is a swept region of the growth chamber. The perfused medium can be any medium suitable for culturing or maintaining plant protoplasts. Suitable protoplast media are known in the art. See, for example, Giles, Kenneth, editor. Plant Protoplasts: International Review of Cytology, Vol. 16, Academic Press 1983; Yoo et al. (2007), Nature Protocols, Vol. 2(7), 1565-72; and Danon (2014), Bio-Protocol, Vol. 4(12), e1149.

The perfused medium may sweep expanded protoplasts out of the connection region of the growth chamber and subsequently out of the microfluidic device. Accordingly, the number of protoplast present in the isolation region of the growth chamber may be stabilized at a maximum number dependent on the size of the protoplasts and size of the isolation region of the growth chamber. The ability to stabilize the maximal number of cells in an isolated population of cells provides an advantage over other currently available methods for cell culturing, as tedious cell population splitting can be eliminated.

In some embodiments, incubating may be carried out for about 1, 2, 3, 4, 5, 6, 7, 8, 9, 10 days, or more. Incubating periods may range from about 1 day to about 6 days, from about 1 day to about 5 days, from about 1 day to about 4 days, from about 1 day to about 3 days, or from about 1 day to about 2 days. In other embodiments incubating may be carried out for less than about 5 days, less than about 4 days, less than about 3 days, or less than about 2 days. In some embodiments, incubating may be carried out for less than about 3 days or less than about 2 days. In other embodiments, incubating may be carried out for about 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, or about 23 h.

During the culturing step, an image of the at least one growth chamber and any cells contained therein may be monitored at one or more time points throughout the culturing step. The image may be stored in the memory of a processing component of the system.

Perfusing the cell. During the incubating step, the second fluidic medium, present within the isolation region of the growth chamber may become depleted of nutrients, growth factors or other growth stimulants. The second fluidic medium may accumulate cellular waste products. Additionally, as the at least one cell (e.g., plant protoplast) continues to grow during the period of incubation, it may be desirable to alter the nutrients, growth factors or other growth stimulants to be different from those of the first or second media at the start of the incubation. Culturing in a growth chamber of a microfluidic device as described here may afford the specific and selective ability to introduce and alter chemical gradients sensed by the at least one plant protoplast, which may much more closely approximate in-vivo conditions. Alternatively, altering the chemical gradients sensed by the at least one biological cell to purposely non-optimized set of conditions may permit cell expansion under conditions designed to explore disease or treatment pathways. The method may therefore include perfusing the first fluidic medium during the incubating step, wherein the first fluidic medium is introduced via at least one inlet 124 of the microfluidic device and wherein the first fluidic medium, optionally comprising components from the second fluidic medium is exported via at least one outlet of the microfluidic device.

Exchange of components of the first fluidic medium, thereby providing fresh nutrients, soluble growth factors, and the like, and/or exchange of waste components of the medium surrounding the cell(s) within the isolation region occurs at the interface of the swept and unswept regions of the growth chamber substantially under conditions of diffusion. Effective exchange has been surprisingly found to result under substantially no flow conditions. Accordingly, it has been surprisingly found that successful incubation does not require constant perfusion. As result, perfusing may be non-continuous. In some embodiments, perfusing is periodic, and in some embodiments, perfusing is irregular. Breaks between periods of perfusion may be of sufficient duration to permit components of the second fluidic medium in the isolation region to diffuse into the first fluidic medium in the flow channel/region and/or components of the first fluidic medium to diffuse into the second fluidic medium, all without substantial flow of the first medium into the isolation region.

In another embodiment, low perfusion rates may also be employed to obtain effective exchange of the components of fluidic media within and outside of the unswept region of the growth chamber.

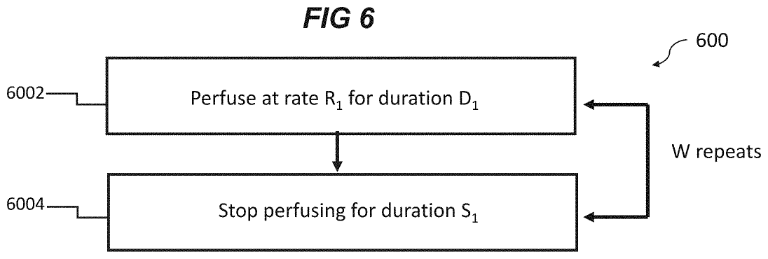

Accordingly, one method of perfusing at least one biological cell in at least one growth chamber of a microfluidic device is shown in FIG. 6 and includes a perfusing step 6002 where the first fluidic medium is flowed into a flow region fluidically connected to the growth chamber at a first perfusion rate R1 for a first perfusion time D1 through a flow region of the microfluidic device. R1 may be selected to be a non-sweeping rate of flow, as described herein. Method 600 further includes the step 6004 of stopping the flow of the fluidic medium for a first perfusion stop time S1. Steps 6002 and 6004 are repeated for W repetitions, where W may be an integer selected from 1 to about 1000, whereupon the perfusion process 700 is complete. In some embodiments, W may be an integer of 2 to about 1000.

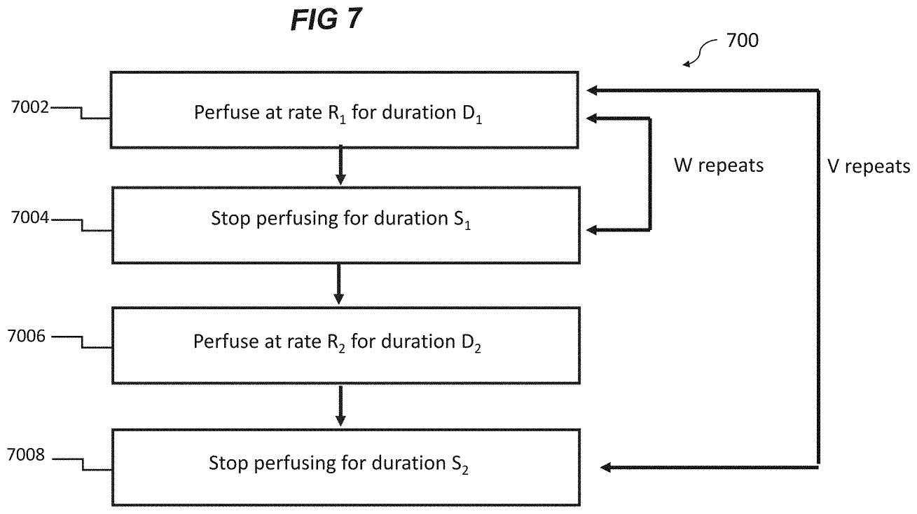

Another method 700, of perfusing at least one biological cell in at least one growth chamber of a microfluidic device is shown in FIG. 7, which includes a first perfusion cycle that includes the step 7002 of flowing the fluidic medium into a flow region fluidically connected to the growth chamber at a first perfusion rate R1 for a first perfusion time D1 through a flow region of the microfluidic device. R1 may be selected to be a non-sweeping rate of flow, as described herein. The first perfusion cycle includes the step 7004 of stopping the flow of the fluidic medium for a first perfusion stop time S1. The first perfusion cycle may be repeated for W repetitions, wherein W is an integer selected from 1 to about 1000. After the Wth repeat of the first perfusion cycle is completed, method 700 further includes a second perfusion cycle, which includes the step 7006 of flowing the first fluidic medium at a second perfusion rate R2 for a second perfusion time D2, wherein R2 is selected to be a non-sweeping rate of flow. The second perfusion cycle of Method 700 further includes the step 7008 of stopping the flow of the fluidic medium for a second perfusion stop time S2. Thereafter, the method returns to step 7002 and 7004 of the first perfusion cycle and the combined two cycle perfusion process is repeated for V repeats, wherein V is an integer of 1 to about 5000. The combination of W and V may be chosen to meet the desired incubation period endpoint.

In various embodiments of method 600, or 700, perfusing rate R1 may be any non-sweeping rate of flow of fluidic medium as described above for flow controller configurations. In some embodiments, R1 may be about 0.009, 0.010, 0.020, 0.030, 0.040, 0.05, 0.06, 0.07, 0.08, 0.09, 0.10, 0.11, 0.12, 0.13, 0.14, 0.15, 0.16, 0.17, 0.18, 0.19, 0.20, 0.30, 0.40, 0.50, 0.60, 0.70, 0.80, 0.90, 1.00, 1.10, 1.20, 1.30, 1.40, 1.50, 1.60, 1.70, 1.80, 1.90, 2.00, 2.10, 2.20, 2.40, 2.50, 2.60, 2.70, 2.80, 2.90 or 3.00 microliters/sec.

In various embodiments of method 700, the second perfusion rate R2 may be any non-sweeping rate of flow of fluidic medium as described as above for flow controller configurations. In some embodiments, the R2 may be 0.009, 0.010, 0.020, 0.030, 0.040, 0.05, 0.06, 0.07, 0.08, 0.09, 0.10, 0.11, 0.12, 0.13, 0.14, 0.15, 0.16, 0.17, 0.18, 0.19, 0.20, 0.30, 0.40, 0.50, 0.60, 0.70, 0.80, 0.90, 1.00, 1.10, 1.20, 1.30, 1.40, 1.50, 1.60, 1.70, 1.80, 1.90, 2.00, 2.10, 2.20, 2.40, 2.50, 2.60, 2.70, 2.80, 2.90 or 3.00 microliters/sec. The flow rates R1 and/or R2 may be chosen in any combination. Typically, perfusion rate R2 may be greater than perfusion rate R1, and may be about 5×, 10×, 20×, 30×, 40×, 50×, 60×, 70×, 80×, 90×, 100×, or more than R1. In some embodiments, R2 is at least ten times faster than R1. In other embodiments, R2 is at least twenty times faster than R1. In yet another embodiment, R2 is at least 100× the rate of R1.

In various embodiments of method 600 or 700, first perfusion time D1 may be any suitable duration of perfusion as described above for flow controller configurations. In various embodiments, D1 may be about 5, 10, 15, 20, 25, 30, 35, 40, 45, 50, 55, 60, 65, 70, 80, 90, 100, 110, 120, 130, 140, 150, 160, 170 or 180 sec. In other embodiments, D1 may be a range of time, e.g., about 10 to about 40 sec, as described above. In some embodiments, D1 may be about 30 sec to about 75 sec. In other embodiments, D1 may be about 100 sec. In other embodiments, D1 may be in a range from about 60 sec to about 150 sec. In yet other embodiments, D1 may be about 20 min, 30 min, 40 min, 50 min, 60 min, 80 min, 90 min, 110 min, 120 min, 140 min, 160 min, 180 min, 200 min, 220 min, 240 min, 250 min, 260 min, 270 min, 290 min or 300 min. In some embodiments, D1 is about 40 min to about 180 min.

In various embodiments of method 600 or 700, second perfusion time D2 may be any suitable duration of perfusion as described above for flow controller configurations. In various embodiments, D2 may be about 5 sec, 10 sec, 15 sec, 20 sec, 25 sec, 30 sec, 35 sec, 40 sec, 45 sec, 50 sec, 55 sec, 60 sec, 65 sec, 70 sec, 80 sec, 90 sec or about 100 sec. In other embodiments, D2 may be a range of time, e.g., about 5 sec to about 20 sec, as described above. In other embodiments, D2 may be about 30 sec to about 70 sec. In other embodiments, D2 may be about 60 sec.

In various embodiments of method 600 or 700, the first perfusion time D1 may be the same or different from the second perfusion time D2. D1 and D2 may be chosen in any combination. In some embodiments, the duration of perfusing D1 and/or D2 may be selected to be shorter than the stopping periods S1 and/or S2.

In various embodiments of method 600 or 700, the first perfusion stop time S1 may be selected to be any suitable period of time as described above for an interval of time between periods of perfusion for flow controller configurations. In some embodiments, S1 may be about 0 min, 5 min, about 10 min, about 15 min, about 20 min, about 25 min, about 30 min, about 35 min, about 40 min, about 45 min, about 60 min, about 65 min, about 80 min, about 90 min, about 100 min, about 120 min, about 150 min, about 180 min, about 210 min, about 240 min, about 270 min, or about 300 min. In various embodiments, S1 may be any appropriate range of time, as described above for flow controller configuration intervals between perfusion, e.g. about 20 to about 60 min. In some embodiments, S1 may be about 10 min to about 30 min. In other embodiments, S1 may be about 15 min. In yet other embodiments, S1 may be about 0 sec, 5 sec, 10 sec, 20 sec, 30 sec, 40 sec, 50 sec, 60 sec, 70 sec, 80 sec, or about 90 sec. In some embodiments, S1 is about 0 sec.

In various embodiments of method 600 or 700, the second perfusion stop time S2 may be selected to be any suitable period of time as described above for an interval of time between periods of perfusion for flow controller configurations. In some embodiments, S2 may be about 0 min, 5 min, about 6 min, about 7 min, about 8 min, about 9 min, about 10 min, about 20 min, about 30 min, about 45 min, about 50 min, about 60 about 90 min, about 120 min, about 180 min, about 240 min, about 270 min, or about 300 min. In various embodiments, S2 may be any appropriate range of time, as described above for flow controller configuration intervals between perfusion, e.g. about 15 to about 45 min. In some embodiments, S2 may be about 10 min to about 30 min. In other embodiments, S2 may be about 8 min or 9 min. In other embodiments, S2 is about 0 min.

In various embodiments of method 600 or 700, the first perfusion stop time S1 and the second perfusion stop time S2 may be selected independently from any suitable value. S1 may be the same or different from S2.

In various embodiments of method 700, the number of W repetitions may be selected to be the same or different from the number of V repetitions.

In various embodiments of methods 600 or 700, W may be about 1, about 4, about 5, about 6, about 8, about 10, about 12, about 15, about 18, about 20, about 24, about 30, about 36, about 40, about 45, or about 50. In some embodiments, W may be selected to be about 1 to about 20. In some embodiments, W may be 1.

In various embodiments of method 700, V may be about 5, about 10, about 20, about 25, about 30, about 35, about 40, about 50, about 60, about 80, about 100, about 120, about 240, about 300, about 350, about 400, about 450, about 500, about 600, about 750, about 900, or about 1000. In some embodiments, V may be selected to be about 10 to about 120. In other embodiments, V may be about 5 to about 24. In some embodiments, V may be about 30 to about 50 or may be about 400 to about 500.

In various embodiments of method 700, the number of W repetitions may be selected to be the same or different from the number of V repetitions.

In various embodiments of methods 600 or 700 a total time for the first step of perfusing (represented by steps 7002/7004 or 8002/8004) is about 1 h to about 10 h and W is an integer is 1. In various embodiments, the total time for the first step of perfusing is about 9 min to about 15 min.

In various embodiments of method 700, a total time for the second step of a perfusing cycle (represented by step 8006/8008) is about 1 min to about 15 min or about 1 min to about 20 min.

In any of methods 600 or 700, the perfusing method may be continued for the entire incubation period of the biological cell, e.g., for about 1, about 2, about 3, about 4, about 5, about 6, about 7, about 8 about 9, about 10 days or more.

In another non-limiting embodiment of method 700 of FIG. 7, the controller may be configured to perfuse the fluidic medium(s) in the flow region having longer periods of perfusion D1 during the perfusing step 7002. The controller may perfuse the fluidic medium at a first rate for a period of about 45 min, about 60 min, about 75 min, about 90 min, about 105 min, about 120 min, about 2.25 h, about 2.5 h, about 2.45 h, about 3.0 h, about 3.25 h, about 3.5 h, about 3.75 h, about 4.0 h, about 4.25 h, about 4.5 h, about 4.75 h, about 5 h, or about 6 h. At the end of the first perfusion period D1, the flow of the fluidic medium may be stopped for a stopping period of time S1, which may be about 0 sec, 15 sec, 30 sec, about 45 sec, about 1 min, about 1.25 min, about 1.5 min, about 2.0 min, about 3.0 min, about 4 min, about 5 min or about 6 min. In some embodiments, the first flow rate R1 may be selected to be about 0.009, 0.01, 0.02, 0.03, 0.05, 0.1, 0.2, 0.3, 0.4, or about 0.5 microliters/sec. The flow of the fluidic medium may be stopped for a perfusion stopping period S1 of less than about 1 minute or S1 may be 0 sec. Alternatively, S1 may be about 30 sec, about 1.5 min, about 2.0 min, about 2.5 min, or about 3 min. A second perfusion period D2 may follow, using a different perfusion rate. In some embodiments, the second perfusion rate may be higher than the first perfusion rate. In some embodiments, the second perfusion rate R2 may be selected from about 1.0, 1.1, 1.2, 1.3, 1.4, 1.5, 1.7, 1.9, 2.0, 2.2, 2.4, 2.6, 2.8, 3.0, 3.2, 3.4, 3.6, 3.8, 4.0, 4.2, 4.4, 4.6, 4.8, 5.0, 6.0, 7.0, 8.0 or about 9.0 microliters/sec. The second perfusion period D2 may be about 1 sec, about 2 sec, about 3 sec, about 4 sec, about 5 sec, about 6 sec, about 10 sec, about 15 sec, about 30 sec, about 45 sec, about 60 sec, about 65 sec, about 75 sec, about 80 sec, or about 90 sec. Perfusing may be then stopped for a second perfusion stop period S2, which may be about 0 sec, 10 sec, about 20 sec, about 30 sec, about 40 sec, about 50 sec, about 60 sec, about 1.5 min, about 1.75 min, about 2.0 min, about 2.5 min, about 2.75 min, about 3.0 min or about 4.0 min. In some embodiments, D1 may be about 2 h, about 3 h, or about 4 h. In various embodiments, D1 may be about 4 h. In various embodiments, S1 may be 0 sec or less than about one minute. The second perfusion period D2 may be about 1 sec to about 6 sec. In some embodiments, the second perfusion stop period S2 may be about 40 sec to about 1.5 min.

Accordingly, a method is provided for perfusing at least one biological cell in at least one growth chamber of a microfluidic device including the steps of: perfusing the at least one biological cell (e.g., plant protoplast) using a first perfusion step including: flowing a first fluidic medium at a first perfusion rate R1 for a first perfusion time D1 through a flow region of the microfluidic device, where the flow region is fluidically connected to the growth chamber, wherein R1 is selected to be a non-sweeping rate of flow; stopping the flow of the first fluidic medium for a first perfusion stop time S1; and repeating the first perfusion step for W repetitions, where W is an integer selected from 1 to 1000. The method may further include a step of perfusing the at least one biological cell using a second perfusion step comprising: flowing the first fluidic medium at a second perfusion rate R2 for a second perfusion time D2, where R2 is selected to be a non-sweeping rate of flow; stopping the flow of the first fluidic medium for a second perfusion stop time S2; and repeating the first perfusion step followed by the second perfusion step for V repetitions, wherein V is an integer of 1 to 1000.

The second perfusion rate R2 may be greater than the first perfusion rate R1. The first perfusion time D1 may be the same or different from the second perfusion time D2. The first perfusion stop time S1 may be the same or different from the second perfusion stop time S2. The number of W repetitions may be the same or different from the number of V repetitions, when the second perfusing step is performed. R2 may be at least ten times faster than R1. Alternatively, R2 may be at least twenty times faster than R1. R2 may be at least 100 times as fast as R1. D1 may be about 30 se to about 75 sec. In other embodiments, D1 may be about 40 min to about 180 min or about 180 min to about 300 min. In some other embodiments, D1 may be about 60 sec to about 150 sec. S1 may be about 10 min to about 30 min. In other embodiments, S1 may be about 5 min to about 10 min. In yet other embodiments, S1 may be zero. In some embodiments, D1 may be about 40 min to about 180 min, and S1 may be zero. In other embodiments, D1 may be about 60 sec to about 150 sec, and S1 may be about 5 min to about 10 min. In yet other embodiments, D1 may be about 180 min to about 300 min, and S1 may be zero. The total time for the first perfusing step may be about 1 h to about 10 h. In other embodiments, the total time for the first perfusing step may be about 2 h to about 4 h. In some embodiments, W may be an integer greater than 2. In some embodiments, W may be about 1 to about 20. In some embodiments, D2 may be about 10 sec to about 25 sec. In other embodiments, D2 may be about 10 sec to about 90 sec. In some embodiments, S2 may be about 10 min to about 30 min. In other embodiments, S2 may be about 15 min. In some embodiments, V may be about 10 to about 120. In some embodiments, V may be about 30 to about 50 or may be about 400 to about 500. In some embodiments, D2 may be about 1 sec to about 6 sec. and S2 may be 0 sec. In some embodiments, D2 may be about 10 sec to about 90 sec and S2 may be about 40 sec to about 1.5 min. In some embodiments, a total time for one repeat of the second perfusing step may be about 1 min to about 15 min.

Conditioning the medium. In order to provide a medium (e.g., first or second medium) that sustains and enhances growth and/or viability for the at least one plant protoplast, the first fluidic medium may contain both liquid and gaseous components (e.g., the gaseous components may be dissolved in the liquid components). In addition, the fluidic medium can include other components, such as biological molecules, vitamins and minerals that are dissolved in the liquid components. Any suitable components may be used in the fluidic media, as is known to one of skill. Some non-limiting examples are discussed above, but many other media compositions may be used without departing from the methods described herein. In some embodiments, the fluidic medium may include a chemically defined medium (at least prior to contacting cells or a cell-containing fluid), and may further be a protein-free or peptide-free chemically defined medium.

The first fluidic medium may be prepared by saturating an initial fluidic medium with dissolved gaseous molecules, prior to introducing the first fluidic medium into the microfluidic device. Additionally, saturating the initial fluidic medium with dissolved gaseous molecules may continue right up to the point in time that the first fluidic medium is introduced into the microfluidic device. Saturating the initial fluidic medium may include contacting the microfluidic device with a gaseous environment capable of saturating the initial fluidic medium with dissolved gaseous molecules. Gaseous molecules that may saturate the initial fluidic medium include but are not limited to oxygen, carbon dioxide and nitrogen.

The first fluidic medium may further include moderating a pH of the first fluidic medium. Moderating the pH of the first fluidic medium can occur, for example, prior to and/or during introduction of dissolved gaseous molecules. Such moderating may be accomplished by the addition of a buffer species. One non-limiting example of a suitable buffering species is HEPES. Other buffering species may be present in the medium and may or may not depend on the presence of carbon dioxide (such as carbonic acid buffer systems), and can be selected by one of skill. Salts, proteins, carbohydrates, lipids, vitamin and other small molecules necessary for cell growth may also form part of the first fluidic medium composition.

In some embodiments, saturating of the first fluidic medium with the gaseous components may be performed in a reservoir prior to introduction via the inlet port. In other embodiments, saturating of the first fluidic medium with the gaseous components may be performed in a gas permeable connecting conduit between the reservoir and the inlet. In yet other embodiments, saturating of the first fluidic medium with the gaseous components may be performed via a gas permeable portion of a lid of the microfluidic device. In some embodiments, the gaseous saturation of the fluidic medium also includes maintaining humidity in the gas exchange environment such that the fluidic medium within the microfluidic device does not change in osmolality during the incubation period.

The composition of the first fluidic medium may also include at least one secreted component from a feeder cell culture. Secreted feeder cell components may include growth factors, hormones, cytokines, small molecules, proteoglycans, and the like. The introduction of the at least one secreted component from the feeder cell culture may be performed in the same reservoir where saturating the first fluidic medium with gaseous components is performed, or introduction of the at least one secreted component from the feeder cell culture to the first fluidic medium may be made prior to the saturating step.

In some other embodiments, the composition of the first medium may also include an additive(s) designed to provide altered fluidic medium to test the response of the cell to the additive(s). Such additive(s) can, for example, increase or decrease cell viability or growth.

In some embodiments, the method may include detecting the pH of the first fluidic medium as it is introduced via the at least one inlet. Detecting the pH may be performed at a location directly proximal to the inlet. In some embodiments, the method may include detecting the pH of the first fluidic medium as the first fluidic medium is exported via an outlet. Detecting the pH may be performed at a location directly proximal to the outlet. Either or both of the detectors used to detect the pH may be an optical sensor. In some embodiments, the detector may be capable of providing an alarm if the pH deviates from an acceptable range. In some other embodiments, when a pH value measured by the detector deviates from an acceptable range, then the composition of the first fluidic medium may be altered.

During the incubating step, an image of the at least one growth chamber and any cells contained therein may be monitored.

Screening plant protoplasts. Plant protoplasts can be screened for disease resistance by contacting the protoplasts with a pathogenic agent or a fragment thereof and monitoring the plant protoplast to determine whether it remains viable. Exemplary screens are described in Example 3, below, and in the Listing of Embodiments and claims. Plant immunity is generally described, for example, in Boutrot and Zipfel (2017), Annu. Rev. Phytopathol. 55:257-86; Boyd et al. (2012), Trends in Genetics, Vol. 29(4), 233-40; and Smith and Heese (2014), Plant Methods, Vol. 10:6. Additionally, screens for pathogen resistance traits are known in the art. See, e.g., Gomez-Gomez and Boller (2000), Molecular Cell, Vol. 5:1003-11; and Steuernagel et al. (2016), Nature Biotechnology, Vol. 34(6), 652-655.

Exporting the at least one biological cell. After the incubating step is complete, the at least one biological cell or colony of cells may be exported out of the growth chamber or the isolation region thereof. Exporting may include using a dielectrophoresis (DEP) force sufficiently strong to move the one or more biological cells/colony of cells. The DEP force may be optically actuated or electronically actuated. For example, the microfluidic device can include a substrate having a DEP configuration, such as an opto-electronic tweezer (OET) configuration. In other embodiments, the at least one biological cell or colony of cells may be exported out of the growth chamber or the isolation region using fluid flow and/or gravity. In yet other embodiments, the at least one biological cell or colony of cells may be exported out of the growth chamber or the isolation region using compressive force on a deformable lid region above the growth chamber or the isolation region thereof, thereby causing a localized flow of fluid out of the growth chamber or isolation region.

After the at least one biological cell or colony of cells is exported out of the growth chamber or the isolation region, then the cells may be exported from the flow region (e.g., channel) out of the microfluidic device. In some embodiments, exporting the cells from the flow region includes using a DEP force sufficiently strong to move the one or more biological cells/colony of cells. The DEP force may be generated as described above. In some other embodiments, exporting the cells from the flow region out of the microfluidic device includes using fluid flow and/or gravity to move the cells.

During the exporting step, an image of the at least one growth chamber and any cells contained therein may be monitored.

Conditioning at least one surface. In some embodiments, the microfluidic device is provided with at least one surface of the at least one growth chamber in a conditioned state. In other embodiments, the surface of the at least one growth chamber is conditioned prior to introducing the at least one biological cell (e.g., plant protoplast) and may be performed as part of the method of culturing the one or more biological cells. Conditioning the surface may include treating the surface with a conditioning reagent, such as a polymer.

In some embodiments, a method is provided for treating at least one surface of at least one growth chamber of a microfluidic device (100, 300, 400, 500A-E, and 600), including the steps of flowing the fluidic medium including an excess of conditioning reagent into the flow channel (FIGS. 1A-1C, 2, 3, 4A-C); incubating the microfluidic device for a selected period of time; and replacing the medium in the channel. In other embodiments, a method for priming a microfluidic device includes the steps of flowing a priming solution containing a conditioning reagent into the flow channel; incubating the device for a selected period of time, thereby conditioning at least one surface of the growth chamber; and replacing the solution in the channel with a fluidic medium. The priming solution may contain any fluidic medium as described herein. The fluidic medium replacing the conditioning solution or the fluidic medium having an excess of conditioning reagent may be any medium as described herein and may additionally contain cells.

In some embodiments, the at least one surface may be treated with a polymeric conditioning reagent including alkylene ether moieties. The polymeric conditioning reagent having alkylene ether moieties may include any suitable alkylene ether containing polymers, including but not limited to any of the alkylene ether containing polymers discussed above. In one embodiment, the surface of the growth chamber may be treated with amphiphilic nonionic block copolymers which include blocks of polyethylene oxide (PEO) and polypropylene oxide (PPO) subunits in differing ratios and locations within the polymer chain (e.g., Pluronic® polymers). Specific Pluronic® polymers useful for yielding a conditioned surface include Pluronic® L44, L64, P85, F68 and F127 (including F127NF).

In other embodiments, the surface may be treated with a polymeric conditioning reagent including carboxylic moieties. Non-limiting examples of suitable carboxylic acid containing polymeric conditioning reagents are discussed above and any appropriate carboxylic acid containing polymeric conditioning reagent may be used to treat the surface.

In other embodiments, the surface may be treated with a polymeric conditioning reagent including saccharide moieties. Non-limiting examples of suitable saccharide containing polymeric conditioning reagents are discussed above and any appropriate saccharide containing polymeric conditioning reagent may be used to treat the surface.

In other embodiments, the surface may be treated with a polymeric conditioning reagent including sulfonic acid moieties. Non-limiting examples of suitable sulfonic acid containing polymeric conditioning reagents are discussed above and any appropriate sulfonic acid containing polymeric conditioning reagent may be used to treat the surface.

In other embodiments, the surface may be treated with a polymeric conditioning reagent including amino acid moieties. Non-limiting examples of suitable amino acid containing polymeric conditioning reagents are discussed above and any appropriate amino acid containing polymeric conditioning reagent may be used to treat the surface. The amino acid containing polymeric conditioning reagent may include a protein.

In other embodiments, the surface may be treated with a polymeric conditioning reagent including nucleic acid moieties. Non-limiting examples of suitable nucleic acid containing polymeric conditioning reagents are discussed above and any appropriate nucleic acid containing polymeric conditioning reagent may be used to treat the surface.

In some embodiments, a mixture of more than one polymeric conditioning reagent may be used to treat the surface of the growth chamber.

In other embodiments, conditioning includes heating the surface of the growth chamber to a temperature of about 30° C. In some embodiments, the method includes heating the surface to a temperature of at least about 20° C., 21° C., 22° C., 23° C., 24° C., 25° C., 26° C., 27° C., 28° C., 29° C., 30° C., 31° C., 32° C., 33° C., 34° C., or about 35° C. In some embodiments, the method includes heating the surface to a temperature of about 25° C. In other embodiments the method includes heating the surface to a temperature in the range from about 20°-30° C.; about 22° C. to about 28° C.; or about 24° C. to about 26° C. In some embodiments, the method includes heating the surface to a temperature of at least about 22° C. In some embodiments, heating the surface includes at least one surface that is conditioned by treating the surface with a polymer.

Clonal population. The methods described here also include methods where only one biological cell (e.g., plant protoplast) is introduced to the at least one growth chamber. A method is provided for cloning a biological cell in a system including a microfluidic device having a flow region configured to contain a flow of a first fluidic medium; and at least one growth chamber including an isolation region and a connection region, the isolation region being fluidically connected with the connection region and the connection region including a proximal opening to the flow region, which includes the steps of introducing the biological cell into the at least one growth chamber, where the at least one growth chamber is configured to have at least one surface conditioned to support cell growth, viability, portability, or any combination thereof, and incubating the biological cell for a period of time at least long enough to expand the biological cell to produce a clonal population of biological cells. In some embodiments, the system may be any system as described herein. The microfluidic device may be any microfluidic device as described herein.

In some embodiments of the method for cloning a biological cell, the at least one conditioned surface may include a linking group covalently linked to the surface, and the linking group may be linked to a moiety configured to support cell growth, viability or portability of the one or more biological cells within the microfluidic device. In some embodiments, the linking group may include a siloxy linking group. In other embodiments, the linking group may include a phosphonate linking group. In some embodiments, the linking group may be indirectly linked to the moiety configured to support cell growth, viability, portability, or any combination thereof. In other embodiments, the linking group may be directly linked to the moiety configured to support cell growth, viability, portability, or any combination thereof. The linking group may be indirectly linked to the moiety configured to support cell growth, viability or movability via connection to a linker. In some embodiments, the linking group may be indirectly linked to the moiety configured to support cell growth, viability or movability via connection to a first end of a linker. In some embodiments, the linker may further include a linear portion wherein a backbone of the linear portion comprises 1 to 200 non-hydrogen atoms selected from any combination of silicon, carbon, nitrogen, oxygen, sulfur and phosphorus atoms. In some embodiments, the backbone of the linear portion may include one or more arylene moieties. In other embodiments, the linker may include a triazolylene moiety. In some embodiments, the triazolylene moiety may interrupt the linear portion of the linker or may be connected at a second end to the linear portion of the linker. In various embodiments, the moiety configured to support cell growth and/or viability and/or portability may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids. In some embodiments, the at least one conditioned surface comprises alkyl or perfluoroalkyl moieties. In other embodiments, the at least one conditioned surface comprises alkylene ether moieties or dextran moieties.

In various embodiments, the method may further include the step of conditioning the at least a surface of the at least one growth chamber. In some embodiments, conditioning includes treating the at least one surface with one or more agents that support cell portability within the microfluidic device. In some embodiments, the conditioning may include treating the at least a surface of the at least one growth chamber with a conditioning reagent including a polymer. In some embodiments, the polymer may include alkylene ether moieties. In some embodiments, the polymer may include carboxylic acid moieties. In some embodiments, the polymer may include saccharide moieties. In other embodiments, the polymer may include sulfonic acid moieties. In yet other embodiments, the polymer may include amino acid moieties. In further embodiments, the polymer may include nucleic acid moieties.

In various embodiments, the conditioning may include heating the at least a surface of the at least one growth chamber to a temperature of about 30° C.

In various embodiments, the method may further include a step of introducing a first fluidic medium into a microfluidic channel of the flow region of the microfluidic device. In some embodiments, introducing the first fluidic medium may be performed prior to introducing the biological cell (e.g., plant protoplast). In some embodiments, introducing the biological cell into the at least one growth chamber may include using a dielectrophoresis (DEP) force having sufficient strength to move the biological cell. In some embodiments, the DEP force may be optically actuated. In some embodiments, the DEP force may be produced by optoelectronic tweezers (OET). In some other embodiments, introducing the biological cell into the at least one growth chamber may include using fluid flow and/or gravity.

In some embodiments, introducing the biological cell into the at least one growth chamber may further include introducing the biological cell into an isolation region of the at least one growth chamber. In some embodiments, the isolation region of the at least one growth chamber may have dimensions sufficient to support cell expansion to no more than 1×102 cells. In some embodiments, the isolation region may be at least substantially filled with a second fluidic medium. In some embodiments, the flow region may be fluidically connected to a proximal opening of a connection region of the at least one growth chamber, and further wherein the connection region may also be fluidically connected to the isolation region of the growth chamber.

In various embodiments, the method may further include a step of perfusing the first fluidic medium during the incubating step, wherein the first fluidic medium may be introduced via at least one inlet port of the microfluidic device and wherein the first fluidic medium, optionally comprising components from the second fluidic medium may be exported via at least one outlet of the microfluidic device. In some embodiments, perfusing may be non-continuous. In some other embodiments, perfusing may be periodic. In yet other embodiments, perfusing may be irregular. In some embodiments, perfusing of the first fluidic medium may be performed at a rate sufficient to permit components of the second fluidic medium in the isolation region diffuse into the first fluidic medium in the flow region and/or components of the first fluidic medium diffuse into the second fluidic medium in the isolation region; and the first medium may not substantially flow into the isolation region. In some embodiments, perfusing the first fluidic medium may be performed for a duration of about 45 sec to about 90 sec about every 10 min to about every 30 min. In some embodiments, perfusing the first fluidic medium may be performed for a duration of about 2 h to about 4 h. In some embodiments, the period of time that the at least one biological cell is incubated may be from about 1 day to about 14 days, or longer.

In some embodiments, a composition of the first fluidic medium may include liquid and gaseous components. In various embodiments, the method may further include a step of saturating the first fluidic medium with dissolved gaseous molecules prior to introducing the first fluidic medium into the microfluidic device. In various embodiments, the method may further include a step of contacting the microfluidic device with a gaseous environment capable of saturating the first fluidic medium or the second fluidic medium with dissolved gaseous molecules. In various embodiments, the method may further include a step of moderating a pH of the first fluidic medium upon introduction of dissolved gaseous molecules. In some embodiments, saturating the first fluidic medium with the gaseous components may be performed in a reservoir prior to introduction via the inlet port, in a gas permeable connector between the reservoir and the inlet port, or via a gas permeable portion of a lid of the microfluidic device. In some embodiments, a composition of the first fluidic medium may include at least one secreted component from a feeder cell culture.

In various embodiments, the method may further include a step of detecting the pH of the first fluidic medium as it is exported via the at least one outlet. In some embodiments, the detecting step may be performed at a location directly proximal to the at the least one outlet. In various embodiments, the method may further include a step of detecting the pH of the first fluidic medium as it is introduced via the at least one inlet port. In some embodiments, the sensor may be an optical sensor. In various embodiments, the method may further include a step of altering a composition of the first fluidic medium.

In various embodiments, the method may further include a step of monitoring an image of the at least one growth chamber and any cells contained therein.

In various embodiments, the biological cell may be a plant cell, such as a protoplast. The plant may be any type of plant, such as a plant used for agriculture, non-limiting examples of which include lettuce, tomato, corn, wheat, tobacco, and the like.

In some embodiments, the biological cell may be a plurality of biological cells and the at least one growth chamber is a plurality of growth chambers. In various embodiments, the method may further include a step of moving no more than one of the plurality of biological cells into each of the plurality of growth chambers.

In some embodiments of the method of cloning a biological cell, the conditioned surface may further include a cleavable moiety. The method may include a step of cleaving the cleavable moiety before exporting one or more biological cells of the clonal population out of the growth chamber or the isolation region thereof.

In various embodiments, the method may further include a step of exporting one or more biological cells of the clonal population out of the growth chamber or the isolation region thereof. In some embodiments, exporting may include using a dielectrophoresis (DEP) force sufficiently strong to move the one or more biological cells. In some embodiments, the DEP force is optically actuated. In some embodiments, the DEP force may be produced by optoelectronic tweezers (OET). In some embodiments, exporting may include using fluid flow and/or gravity. In some embodiments, exporting may include using compressive force on a deformable lid region above the growth chamber or the isolation region thereof. In various embodiments, the method may further include a step of exporting one or more biological cells of the clonal population from the flow region out of the microfluidic device. In some embodiments, exporting may include using a DEP force sufficiently strong to move the one or more biological cells. In some embodiments, the DEP force is optically actuated. In some embodiments, the DEP force may be produced by optoelectronic tweezers (OET). In some embodiments, exporting may include using fluid flow and/or gravity.

Kits. Kits may be provided for culturing and screening a plant cell, particularly a plant protoplast, where the kit includes: a microfluidic device having a flow region configured to contain a flow of a first fluidic medium and at least one growth chamber; a surface conditioning reagent; and an assay reagent. In this embodiment, the at least one growth chamber has not been pre-treated to condition the at least one surface of the at least one growth chamber, and the conditioned surface is created by treating with the surface conditioning reagent before cell(s) are introduced. Other kits for culturing a plant cells (e.g., plant protoplast) are also provided, where the kit includes a microfluidic device having a flow region configured to contain a flow of a first fluidic medium; and at least one growth chamber comprising an isolation region and a connection region, wherein the isolation region is fluidically connected with the connection region and the connection region comprises a proximal opening to the flow region; a surface conditioning reagent; and an assay reagent, wherein the surface conditioning reagent, when applied to an internal surface of the microfluidic device, generates a surface that support cell growth, viability, portability, or any combination thereof. Yet other kits are provided for culturing and screening a plant cell, such as a plant protoplast, which include: a microfluidic device including a flow region configured to contain a flow of a first fluidic medium, and at least one growth chamber including an isolation region and a connection region, in which the isolation region is fluidically connected with the connection region and the connection region has a proximal opening to the flow region, and the at least one growth chamber has at least one surface comprising a covalently bound a surface modifying ligand; a surface. Alternatively, kits may be provided for culturing a biological cell, where the kit includes: a microfluidic device having a flow region configured to contain a flow of a first fluidic medium; and at least one growth chamber having at least one conditioned surface which can support cell growth, viability, portability, or any combination thereof, and a surface conditioning reagent. The microfluidic device of any of the kits may be any one of microfluidic devices 100, 200, 240, 290, 400, 500A-E, or 600 and have any of the features described above.

The microfluidic device of any of the kits may further include a microfluidic channel including at least a portion of the flow region, and the device may further include a growth chamber having a connection region that opens directly into the microfluidic channel. The growth chamber may further include an isolation region. The isolation region may be fluidically connected to the connection region and may be configured to contain a second fluidic medium, where when the flow region and the at least one growth chamber are substantially filled with a first and second fluidic media respectively, then components of the second fluidic medium diffuse into the first fluidic medium and/or components of the first fluidic medium diffuse into the second fluidic medium; and the first medium does not substantially flow into the isolation region.

In various embodiments of any of the kits, growth chambers may be configured like growth chambers 124, 126, 128, 130, 244, 246, 248, or 436 of FIGS. 1A-1C, 2, 3 and 4A-4C where the isolation region of the growth chamber may have a volume configured to support no more than about 1×10³, 5×10², 4×10², 3×10², 2×10², 1×10², 50, 25, 15, or 10 cells in culture. In other embodiments, the isolation region of the growth chamber has a volume that can support up to about 10, 50 or 1×10² cells. Any configuration of the growth chambers as discussed above may be used in the growth chambers of the microfluidic devices of the kits.

In various embodiments of any of the kits, the size of the growth chambers may be configured to maintain no more than 1×10² biological cells, where the volume of the growth chambers may be no more than 1×10⁷ cubic microns. In other embodiments, wherein no more than 1×10² biological cells may be maintained, the volume of the growth chambers may be no more than 5×10⁶ cubic microns. In yet other embodiments, no more than 50 biological cells may be maintained, and the volume of the growth chambers may be no more than 1×10⁶ cubic microns, or no more than 5×10⁵ cubic microns. In the kits, the microfluidic devices may have any number of growth chambers as discussed above.

The microfluidic device of any of the kits may further include at least one inlet port configured to input the fluidic medium (e.g., first or second fluidic medium) into the flow region and at least one outlet configured to receive the fluidic medium (e.g., spent first fluidic medium), as it exits from the flow region.

The microfluidic device of any of the kits may further include a substrate having a plurality of DEP electrodes, where a surface of the substrate forms a surface of the growth chamber and the flow region. The plurality of DEP electrodes may be configured to generate a dielectrophoresis (DEP) force sufficiently strong to move one or more biological cells (e.g., a clonal population) into or to move one or more cells of a biological cell culture out of a growth chamber or the isolation region thereof. The DEP electrodes, and thus the DEP force may be optically actuated. Such optically actuated DEP electrodes may be virtual electrodes (e.g., regions of an amorphous silicon substrate having increased conductivity due to incident light), phototransistors, or electrodes switched on or off by a corresponding phototransistor. Alternatively, the DEP electrode and thus the DEP force, may be electrically actuated. In some other embodiments, the microfluidic device may further include a substrate having a plurality of transistors, wherein a surface of the substrate forms a surface of the growth chamber and the flow region. The plurality of transistors may be capable of generating a dielectrophoresis (DEP) force sufficiently strong to introduce the biological cell or to move one or more cells of a biological cell culture out of the growth chamber or the isolation region thereof. Each of the plurality of transistors may be optically actuated, and the DEP force may be produced by optoelectronic tweezers.

The microfluidic device of any of the kits may further include a deformable lid region above the at least one growth chamber or isolation region thereof, whereby depressing the deformable lid region exerts a force to export one or more biological cells (e.g., a clonal population) from the growth region to the flow region.

The microfluidic device of any of the kits may be configured to have a lid which is substantially impermeable to gas. Alternatively, all of a portion of the lid may be configured to be gas permeable. The permeable portion of the lid may be permeable to at least one of carbon dioxide, oxygen, and nitrogen. In some embodiments, the lid (or a portion thereof) may be permeable to a combination of more than one of carbon dioxide, oxygen, or nitrogen.

Any of the kits may further include a reservoir configured to contain a fluidic medium. The reservoir may be fluidically connected to any of the microfluidic devices described herein. The reservoir may be configured such that the fluidic medium present in the reservoir may be contacted by a gaseous environment capable of saturating the fluidic medium with dissolved gaseous molecules. The reservoir may further be configured to contain a population of feeder cells in fluidic contact with the fluidic medium.

Any of the kits may include at least one connecting conduit configured to be connected to an inlet port and/or outlet port of the microfluidic device. The connecting conduit may also be configured to connect to a reservoir or a flow controller, such as a pump component. The connecting conduit may be gas permeable. The gas permeable connecting conduit may be permeable to at least one of carbon dioxide, oxygen, and nitrogen. In some embodiments, the gas permeable conduit may be permeable to a combination of more than one of carbon dioxide, oxygen, or nitrogen.

Any of the kits may further include a sensor configured to detect a pH of a first fluidic medium. The sensor may be connected to (or connectable to) an inlet port of the microfluidic device or a connecting conduit attached thereto. Alternatively, the sensor may be integral to the microfluidic device. The sensor may be connected proximal to the point at which fluidic medium enters the microfluidic device. The kit may include a sensor configured to detect a pH of fluidic medium at the outlet of the microfluidic device. The sensor may be connected to (or connectable to) an outlet port of the microfluidic device or a connecting conduit attached thereto. Alternatively, the sensor may be integral to the microfluidic device. The sensor may be connected proximal to the point at which fluidic medium exits the microfluidic device. The sensor, whether attached to the inlet and/or the outlet of the microfluidic device, may be an optical sensor. An optical sensor may include a LED and an integrated colorimetric sensor, which may optionally be a color-sensitive phototransistor. The kit may further include driving electronic components to control the pH sensor and to receive output therefrom. The kit may further include a pH detection reagent. The pH detection reagent may be a pH-sensitive dye that may be detected under visible light.

Any of the kits may also include a culture medium having components capable of enhancing biological cell viability on the microfluidic device. These components may be any suitable culture medium components as is known in the art, including any of the components discussed above for fluidic media components.

Any of the kits may further include at least one reagent to detect a status of a biological cell or a population of cells. Reagents configured to detect the status of the cell are well known in the art, and may be used, for example, to detect whether a cell is alive or dead; is secreting a substance of interest such as antibodies, cytokines, or grow factors; or has cell surface markers of interest. Such reagents may be used without limitation in the kits and methods described herein.

For any of the kits provided herein, the components of the kits may be in separate containers. For any of the components of the kits provided in solution, the components may be present in a concentration that is about 1×, 5×, 10×, 100×, or about 1000× the concentration as used in the methods of the disclosure.

For the kits where the at least one growth chamber of the microfluidic device has not been pre-treated to condition the at least one surface of the at least one growth chamber, and where the conditioned surface is created by treating with the surface conditioning reagent or for kits including a microfluidic device having a flow region configured to contain a flow of a first fluidic medium; and at least one growth chamber having at least one conditioned surface which can support cell growth, viability, portability, or any combination thereof, and a surface conditioning reagent, the surface of the growth chamber may be pre-conditioned with a surface conditioning reagent. The surface conditioning reagent may include a polymer, which may be any one or more of the polymers described above for use as a surface conditioning reagent. In some embodiments, the surface conditioning reagent may include a polymer having alkylene ether moieties, carboxylic acid moieties, sulfonic acid moieties, amino acid moieties, nucleic acid moieties, saccharide moieties, or any combination thereof. The surface conditioning reagent may include a PEO-PPO block co-polymer, such as a Pluronic® polymer (e.g., L44, L64, P85 or F127.

Alternatively, the surface conditioning reagent used to condition the surface of the growth chamber may be included in the kit, separate from the microfluidic device. In other embodiments of the kit, a pre-conditioned microfluidic device is included along with a surface conditioning reagent different from that used to condition the surface of the growth chamber. The different surface conditioning reagent may be any of the surface conditioning reagents discussed above. In some embodiments, more than one surface conditioning reagent is included in the kit.

In various embodiments of the kits having a microfluidic device where the at least one growth chamber of the microfluidic device has not been pre-treated to condition the at least one surface, the kit may also include a culture medium suitable for culturing the one or more biological cells. In some embodiments, the kit may also include a culture medium additive comprising a reagent capable of replenishing the conditioning of a surface of the growth chamber. The culture medium additive may include a conditioning reagent as discussed above or another chemical species enhancing the ability of the at least one surface of the at least one growth chamber to support cell growth, viability, portability, or any combination thereof. This can include growth factors, hormones, antioxidants or vitamins, and the like.

The kit may also include a flow controller configured to perfuse at least the first fluidic medium, which may be a separate component of the microfluidic device or may be incorporated as part of the microfluidic device. The controller may be configured to perfuse the fluidic medium non-continuously. Thus, the controller may be configured to perfuse the fluidic medium in a periodic manner or in an irregular manner.

In another aspect, a kit is provided for culturing a biological cell (e.g., plant protoplast), including a microfluidic device having a flow region configured to contain a flow of a first fluidic medium; and at least one growth chamber comprising an isolation region and a connection region, wherein the isolation region is fluidically connected with the connection region and the connection region comprises a proximal opening to the flow region; and further wherein the at least one growth chamber comprises at least one surface conditioned to support cell growth, viability, portability, or any combination thereof. The microfluidic device may be any microfluidic device as described herein, and may have any of the growth chambers as described herein. The microfluidic device may have a substrate having a DEP configuration of any kind described herein. The DEP configuration may be optically actuated. The substrate of the microfluidic device may have a surface including the substrate compositions as described herein of Formula 1 or Formula 2, and have all the features as described above.

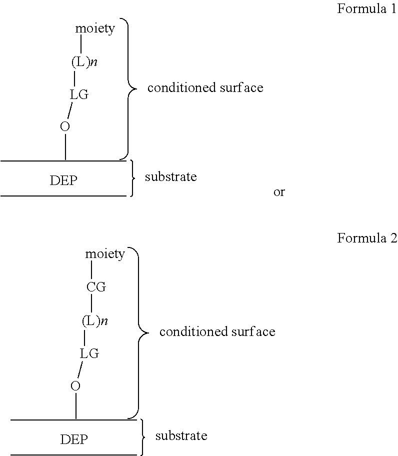

The at least one conditioned surface of the microfluidic device of the kit may include saccharide moieties, alkylene ether moieties, amino acid moieties, alkyl moieties, fluoroalkyl moieties (which may include perfluoroalkyl moieties), anionic moieties, cationic moieties, and/or zwitterionic moieties. In some embodiments, the conditioned surface of the microfluidic device may include saccharide moieties, alkylene ether moieties, alkyl moieties, fluoroalkyl moieties, or amino acid moieties. The alkyl or perfluoroalkyl moieties may have a backbone chain length of greater than 10 carbons. In some embodiments, the conditioned surface to support cell growth, viability, portability, or any combination thereof may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaine; sulfamic acid; or amino acids.

In some embodiments of the kit, the conditioned surface may include a linking group covalently linked to a surface of the microfluidic device, and the linking group may be linked to the moiety configured to support cell growth, viability, portability, or any combination thereof, of the one or more biological cells within the microfluidic device. The linking group may be a siloxy linking group. Alternatively, the linking group may be a phosphonate ester linking group. In some embodiments of the kit, the linking group of the conditioned surface may be directly linked to the moiety configured to support cell growth, viability, portability or any combination thereof.

In other embodiments, the linking group may be indirectly linked to the moiety configured to support cell growth, viability, portability or any combination thereof via a linker. The linking group may be indirectly linked to the moiety configured to support cell growth, viability, portability, or any combination thereof, via connection to a first end of a linker. The linker may further include a linear portion wherein a backbone of the linear portion comprises 1 to 200 non-hydrogen atoms selected from any combination of silicon, carbon, nitrogen, oxygen, sulfur and phosphorus atoms. In some embodiments of the kit, the linker of the conditioned surface may further include a triazolylene moiety. The cleavable moiety is configured to permit disruption of the conditioned surface thereby promoting portability of the biological cell. The kit may further include a reagent configured to cleave the cleavable moiety of the conditioned surface.

In various embodiments of the kit, the kit may further include a surface conditioning reagent. In some embodiments, the surface conditioning reagent may include a polymer comprising at least one of alkylene ether moieties, carboxylic acid moieties, sulfonic acid moieties, phosphonic acid moieties, amino acid moieties, nucleic acid moieties or saccharide moieties. In some other embodiments, the surface conditioning reagent comprises a polymer comprising at least one of alkylene ether moieties, amino acid moieties, or saccharide moieties. In some other embodiments, the conditioned surface may include a cleavable moiety.

In other embodiments of the kit, the surface conditioning reagent comprises at least one cell adhesion blocking molecule. In some embodiments, the at least one cell adhesion blocking molecule may disrupt actin filament formation, block integrin receptors, or reduce binding of cells to DNA fouled surfaces. In some embodiments, the at least one cell adhesion blocking molecule may be Cytochalasin B, an RGD containing peptide, a DNase 1 protein, a fibronectin inhibitor, or an antibody to an integrin. In some embodiments, the at least one cell adhesion blocking molecule may include a combination of more than one type of cell adhesion blocking molecules.

In various embodiments of the kit, the kit may further include a culture medium suitable for culturing the one or more biological cells. In some embodiments, the kit may include a culture medium additive including a reagent configured to replenish the conditioning of the at least one surface of growth chamber. The culture medium additive may include a conditioning reagent as discussed above or another chemical species enhancing the ability of the at least one surface of the at least one growth chamber to support cell growth, viability, portability, or any combination thereof. This can include growth factors, hormones, antioxidants or vitamins, and the like.

In various embodiments of the kit, the kit may include at least one reagent to detect a status of the one or more biological cells.

In yet another aspect, a kit for culturing a biological cell, including a microfluidic device for culturing one or more biological cells including a flow region configured to contain a flow of a first fluidic medium; and at least one growth chamber including an isolation region and a connection region, wherein the isolation region is fluidically connected with the connection region and the connection region has a proximal opening to the flow region; and the at least one growth chamber has at least one surface having a surface modifying ligand. The microfluidic device may be any microfluidic device as described herein. The surface may include a substrate having a dielectrophoresis (DEP) configuration. The DEP configuration may be any DEP configuration described herein. The DEP configuration may be optically actuated. The substrate is any substrate having a surface modifying ligand as described herein, and may have a structure of Formula 3, and may include all the features as described above:

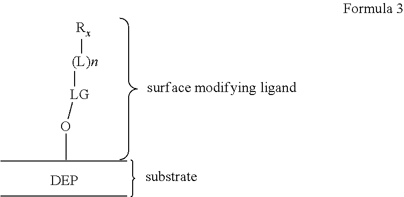

In various embodiments of the kit having a microfluidic device having at least one surface including a surface modifying ligand, the surface modifying ligand may be covalently linked to oxide moieties of the surface of the substrate. The surface modifying ligand may include a reactive moiety. The reactive moiety of the surface modifying ligand may be azido, amino, bromo, a thiol, an activated ester, a succinimidyl or alkynyl moiety. The surface modifying ligand may be covalently linked to the oxide moieties via a linking group. In some embodiments, the linking group may be a siloxy moiety. In other embodiments, the linking group may be a phosphonate ester moiety. The linking group may be connected indirectly via a linker to the reactive moiety of the surface modifying ligand. The linker may include a linear portion wherein a backbone of the linear portion comprises 1 to 100 non-hydrogen atoms selected from any combination of silicon, carbon, nitrogen, oxygen, sulfur and phosphorus atoms. In some embodiments, the surface modifying ligand may include one or more cleavable moieties. The one or more cleavable moieties may be configured to permit disruption of a conditioned surface of a microfluidic device once formed, thereby promoting portability of the one or more biological cells after culturing.

In some embodiments of the kit having a microfluidic device having at least one surface including a surface modifying ligand, the kit may further include a conditioning modification reagent including a first moiety configured to support cell growth, viability, portability, or any combination thereof, and a second moiety configured to react with the reactive moiety of the surface modifying ligand, which may have a structure of Formula 5, and have any of the features as described herein:

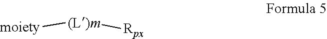

The second moiety may be configured to convert the surface modifying ligand into a conditioned surface configured to support cell growth, viability, portability, or any combination thereof, of one or more biological cells within the growth chamber upon reaction with the reactive moiety of the surface modifying ligand of the microfluidic device of the kit. The first moiety may include an alkylene oxide moiety, a saccharide moiety; an alkyl moiety, a perfluoroalkyl moiety, an amino acid moiety, an anionic moiety, a cationic moiety or a zwitterionic moiety. In some embodiments, the first moiety may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaine; sulfamic acid; or amino acids. The second moiety may be an amino, carboxylic acid, alkyne, azide, aldehyde, bromo, or thiol moiety. In some embodiments, the first moiety or a linker L′ (as described above for Formula 5) of the conditioning modification reagent may include a cleavable moiety. The cleavable moiety may be configured to permit disruption of the conditioned surface thereby promoting portability of the biological cell. In some embodiments, the kit may further include a reagent configured to cleave the cleavable moiety of the conditioned surface.

In some embodiments of the kit having a microfluidic device having at least one surface including a surface modifying ligand, the kit may further include a surface conditioning reagent.

In some embodiments of the kit having a microfluidic device having at least one surface including a surface modifying ligand, the surface conditioning reagent may include a polymer comprising at least one of alkylene ether moieties, carboxylic acid moieties, sulfonic acid moieties, phosphonic acid moieties, amino acid moieties, nucleic acid moieties or saccharide moieties. In some other embodiments, the surface conditioning reagent comprises a polymer comprising at least one of alkylene ether moieties, amino acid moieties, or saccharide moieties. In some other embodiments, the conditioned surface may include a cleavable moiety.

In some embodiments of the kit having a microfluidic device having at least one surface including a surface modifying ligand, the surface conditioning reagent comprises at least one cell adhesion blocking molecule. In some embodiments, the at least one cell adhesion blocking molecule may disrupt actin filament formation, block integrin receptors, or reduce binding of cells to DNA fouled surfaces. In some embodiments, the at least one cell adhesion blocking molecule may be Cytochalasin B, an RGD containing peptide, a DNase 1 protein, a fibronectin inhibitor, or an antibody to an integrin. In some embodiments, the at least one cell adhesion blocking molecule may include a combination of more than one type of cell adhesion blocking molecules.

In some embodiments of the kit having a microfluidic device having at least one surface including a surface modifying ligand, the kit may further include a culture medium suitable for culturing the one or more biological cells. In some embodiments, the kit may further include a culture medium additive including a reagent configured to replenish the conditioning of the at least one surface of growth chamber. The culture medium additive may include a conditioning reagent as discussed above or another chemical species enhancing the ability of the at least one surface of the at least one growth chamber to support cell growth, viability, portability, or any combination thereof. This can include growth factors, hormones, antioxidants or vitamins, and the like.

In some embodiments of the kit having a microfluidic device having at least one surface including a surface modifying ligand, the kit may further include at least one reagent to detect a status of the one or more biological cells.

### EXAMPLES

### Example 1. Culturing and Growth of Grape and Lettuce Protoplasts in a Microfluidic Device

System and Microfluidic device: The system included a Beacon® instrument (Berkeley Lights, Inc.) and OptoSelect™ 3500 and 1750 microfluidic chips (Berkeley Lights, Inc). The instrument comprised a flow controller, temperature controller, fluidic medium conditioning and pump component, structure light source for light activated DEP configurations, mounting stage/nest, and a camera. The microfluidic chips included a substrate having a phototransistor array resting on a first electrode and a cover having an ITO electrode on its inner surface; a silicone microfluidic circuit material was sandwiched between the substrate and cover, and collectively with the substrate and cover defined a microfluidic circuit comprising an inlet, and outlet, a plurality of microfluidic channels. The OptoSelect™ 3500 chips include approximately 3500 sequestration pens, each pen having a volume of about 5×10⁵ cubic microns (i.e., ˜0.5 nL); the OptoSelect™ 1750 chips include approximately 1750 sequestration pens, each pen having a volume of about 1.1×10⁶ cubic microns (i.e., ˜1.1 nL). The internal surfaces of the microfluidic chips included a coating of covalently linked polyethylene (PEG) polymers.

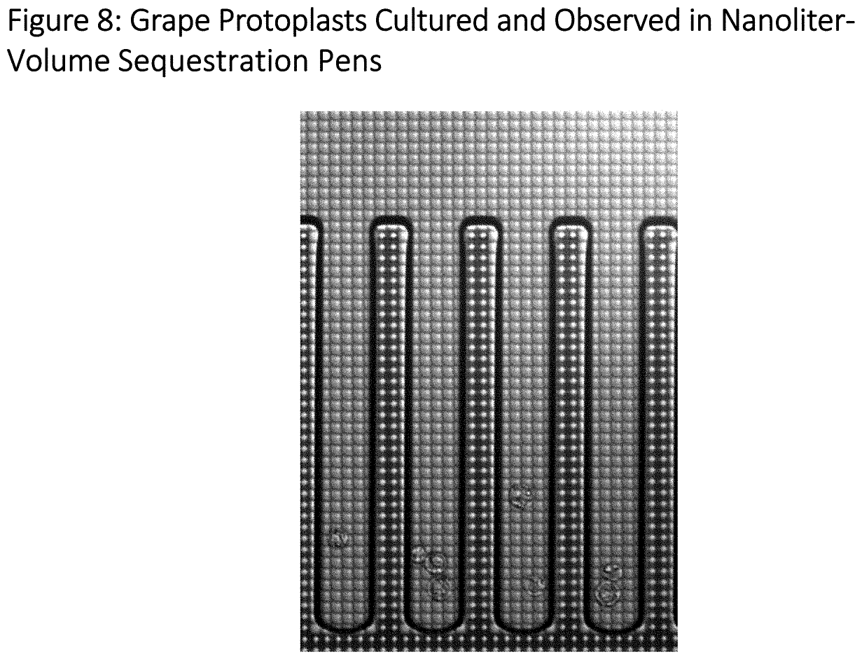

First, grape protoplasts were prepared according to standard procedures, loaded into an OptoSelect™ 3500 microfluidic chip in standard protoplast medium, introduced into growth chambers (in this case, sequestration pens) using gravity (i.e., by standing the microfluidic chip on its side for a period of time), then incubated (i.e., cultured) in standard protoplast medium for a period of approximately 48 hours, with continuous perfusion. The protoplast displayed continued viability over the course of the experiment, as determined by time lapse imaging showing continuous movement of internal structures within the protoplasts. FIG. 8 shows a brightfield image of the grape protoplasts taken during the experiment, with 1 to 3 protoplasts in each of the sequestration pens shown. As an alternative to gravity load, the grape protoplasts could have been loaded into the sequestration pens using DEP force (e.g., light-activated DEP, or OEP™).

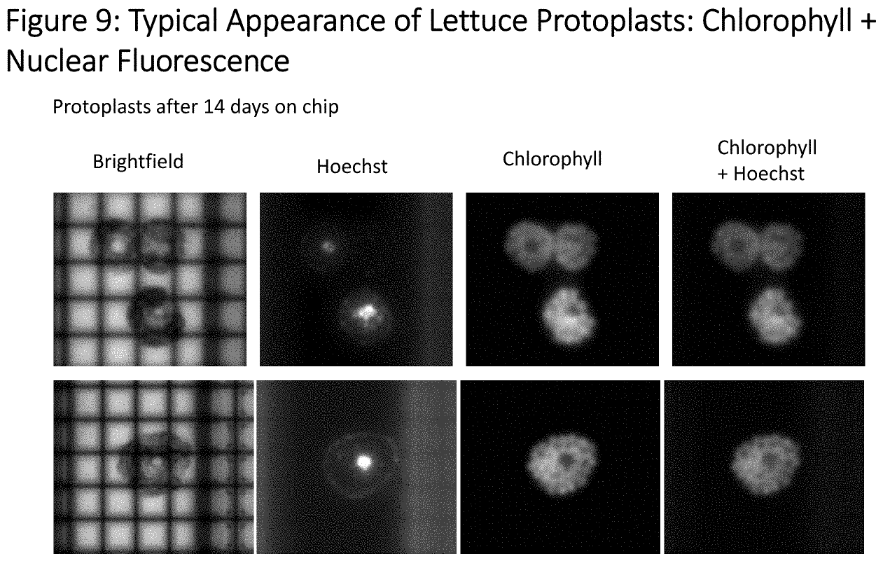

Next, lettuce protoplasts were prepared according to standard procedures, loaded into OptoSelect™ 1750 microfluidic chips in standard protoplast medium, introduced into growth chambers (in this case, sequestration pens) using gravity (i.e., by standing the microfluidic chip on its side for a period of time), then cultured in standard protoplast medium for a period of approximately 14 days, with intermittent perfusion of fresh protoplast medium (including fluorescently labeled dyes, as discussed below) occurring every third day. During the culture period, the lettuce protoplasts were stained with various dyes, including (i) fluorescein diacetate, to detect cell viability, (ii) a chlorophyll stain, (iii) calcofluor white, to detect cell walls, and (iv) Hoechst, to detect nuclei. FIG. 9 shows exemplary images of two different sequestration pens containing lettuce protoplasts at the end of the fourteen-day culture period, including brightfield images and fluorescent images of the protoplasts stained with Hoechst, the chlorophyll stain, or merged images with the Hoechst and chlorophyll stains. Images of the fluorescein and calcofluor white stains (not shown) revealed that cell wall rebuilding correlated with viability, as expected.

As an alternative to gravity load, experiments with lettuce protoplasts were performed in which the protoplasts were loaded into the sequestration pens using DEP force (e.g., light-activated DEP, or OEP™). Using standard DEP force settings used for mammalian cells, a high percentage of penning (>90%) was achieved. Moreover, the viability of the DEP-penned lettuce protoplasts showed no evident change relative to the gravity-loaded protoplasts.

During the fourteen-day culture period, the lettuce protoplasts started to adhere to the surface of the sequestration pens. To export and recover the protoplasts from the microfluidic chips, a mild laser treatment (i.e., 40% power for 400 milliseconds) was applied to the surface of the substrate in the distal right corner of the sequestration pens. The laser treatment dislodges the protoplasts sufficient to allow DEP force to be used to selectively export protoplast clones and recover them off chip. Following export, the protoplast clones can be processed by standard methods to regenerate complete plants.

### Example 2. Genotyping Protoplasts

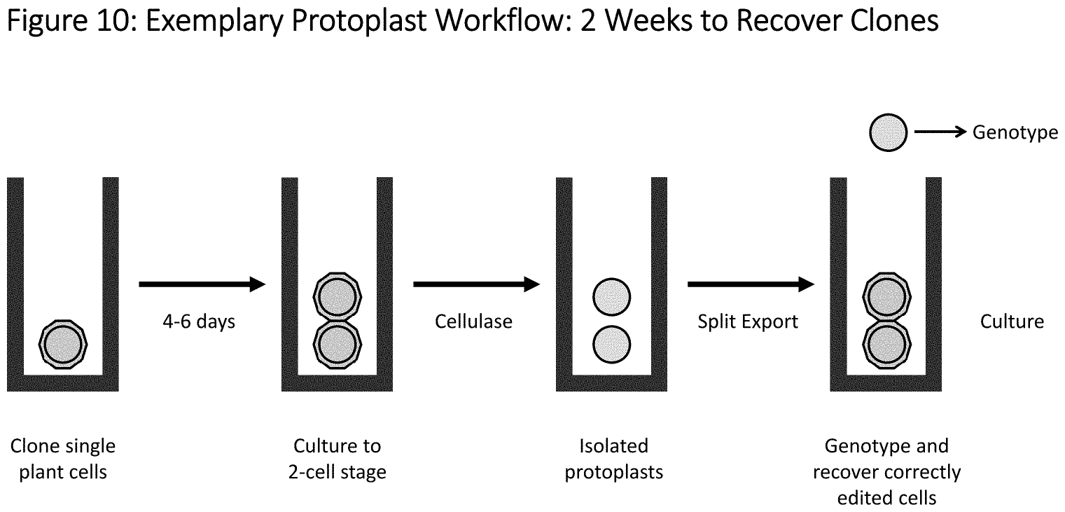

Plant protoplasts are cultured in a microfluidic device to generate clonal colonies, essentially as described in Example 1, above. The protoplasts can be grape protoplasts, lettuce protoplasts, or any other plant protoplast described herein. After formation of colonies, cellulase is perfused in with fresh culture medium, then incubated for sufficient time to allow the protoplasts to separate from one another (this can be visually monitored to confirm separation). For each of one or more select protoplast colonies (e.g., selected based on viability markers and/or appearance), a sub-set of the protoplasts in the colony is individually exported from its corresponding sequestration pen using DEP force, optionally proceeded by application of a laser pulse. The exported cells are then recovered off-chip in standard tissue culture plates by flowing medium containing the exported protoplasts out of the microfluidic chip. The exported protoplasts are processed to obtain nucleic acids (e.g., RNA for transcriptome analysis and/or DNA for genomic analysis), which is sequenced and used to genotype the viable protoplasts from the colony remaining on chip. FIG. 10 provides a schematic diagram of this workflow.

### Example 3. Identification of Disease-Resistance Traits in a Microfluidic Device

Plant protoplasts are cultured in a microfluidic device to generate clonal colonies, essentially as described in Example 1, above. The protoplasts can be grape protoplasts, lettuce protoplasts, or any other plant protoplast described herein.

After culturing the protoplasts for a first period of time, the protoplasts are exposed to/contacted with a pathogenic agent for a second period of time. The pathogenic agent can be the pathogen itself, such as a virus, a bacterial cell, a fungal cell, or the like. Alternatively, the pathogenic agent can be a portion of the pathogen that has the ability to trigger immunity in plants. For example, the pathogen can be a flagellar protein (e.g., a bacterial flagellar protein), a lipopolysaccharide (e.g., LPS A), a peptide glycan, a chitin protein, a capsid protein (e.g., a viral capsid protein), or the like. To contact the protoplasts with the pathogenic agent, the pathogenic agent is flowed into the microfluidic device and allowed to diffuse into the sequestration pens where it can contact the surface of the protoplasts. Alternatively, after being glowed into the microfluidic device, the pathogenic agent can be actively moved into the sequestration pens using a force, such as DEP, localized flow, or the like. Active movement of the pathogenic agent tends to work better with intact pathogens, whereas passive movement of the pathogenic agent tends to work better with molecular agents.

During the second period of time, protoplasts are monitored for changes in viability. Viability can be monitored by brightfield observations, fluorescent viability stains (e.g., fluorescein diacetate, Hoechst, calcofluor white, a chlorophyll stain, or the like). If the plant protoplast has resistance to the pathogen, exposure to the pathogen will induce a cell death pathway in the protoplast, resulting in an observable decrease in viability. If, however, the protoplast remains viable after being contacted with the pathogenic agent, then it can be exported for genotyping, to identify the genetic origin of the lack of pathogen resistance. The genotyping can be focused, for example, on known plant immunity genes, such as Effector Triggered Immunity (ETI) genes, Effector Triggered Susceptibility (ETS) genes, and/or Pathogen Associated Molecular Pattern (PAMP) genes and, optionally, the known plant immunity genes can be selected based on the pathogenic agent to which the protoplasts are exposed.

Prior to introducing the protoplasts into the microfluidic device, the protoplasts can be treated with a mutagen (e.g., a chemical mutagen or transfected with a nucleic acid targeting construct, such as a gene editing construct). Alternatively, the protoplast can be mutagenized on chip, by flowing the mutagen into the microfluidic device and contacting the protoplasts within sequestration pens with the mutagen (e.g., by allowing the mutagen to diffuse into the sequestration pens, towards the protoplasts).

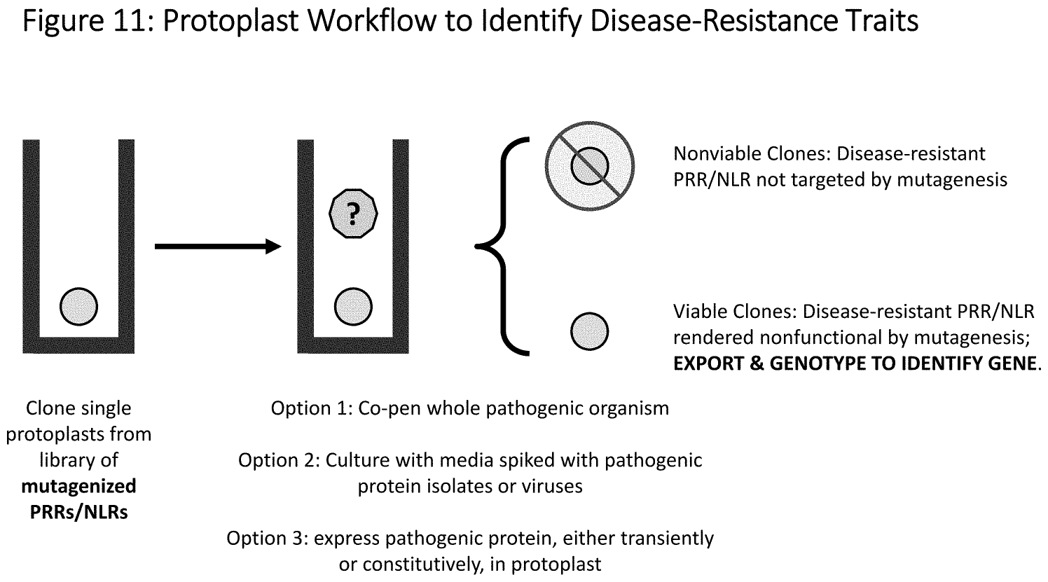

FIG. 11 provides a schematic diagram of the foregoing workflow for identifying disease-resistant traits.

The examples described herein are exemplary in nature and in no way intended to limit the scope of the methods and kits described throughout the entire description.

### LISTING OF EMBODIMENTS

Embodiment 1. A microfluidic device for culturing one or more plant protoplasts, the device comprising: a flow region configured to contain a flow of a first fluidic medium; and at least one growth chamber comprising an isolation region and a connection region, the isolation region being fluidically connected with the connection region and the connection region comprising a proximal opening to the flow region, wherein the at least one growth chamber further comprises at least one surface conditioned to support cell growth, viability, portability, or any combination thereof within the microfluidic device.

Embodiment 2. The microfluidic device of embodiment 1, wherein the at least one conditioned surface is conditioned with one or more agents that support cell portability within the microfluidic device.

Embodiment 3. The microfluidic device of embodiment 1 or 2, wherein the at least one conditioned surface is conditioned with a polymer comprising alkylene ether moieties.

Embodiment 4. The microfluidic device of any one of embodiment 1 to 3, wherein the at least one conditioned surface is conditioned with a polymer comprising saccharide moieties.

Embodiment 5. The microfluidic device of any one of embodiments 1 to 4, wherein the at least one conditioned surface is conditioned with a polymer comprising amino acid moieties.

Embodiment 6. The microfluidic device of any one of embodiments 1 to 5, wherein the at least one conditioned surface of the microfluidic device is conditioned with a polymer comprising carboxylic acid moieties, sulfonic acid moieties, nucleic acid moieties, or phosphonic acid moieties.

Embodiment 7. The microfluidic device of any one of embodiments 1 to 6, wherein the at least one conditioned surface comprises a linking group covalently linked to a surface of the microfluidic device, and wherein the linking group is linked to a moiety configured to support cell growth, viability, portability, or any combination thereof within the microfluidic device.

Embodiment 8. The microfluidic device of embodiment 7, wherein the linking group is a siloxy linking group.

Embodiment 9. The microfluidic device of embodiment 7 or 8, wherein the at least one conditioned surface comprises alkyl or fluoroalkyl moieties.

Embodiment 10. The microfluidic device of embodiment 9, wherein the alkyl or fluoroalkyl moieties have a backbone chain length of greater than 10 carbons.

Embodiment 11. The microfluidic device of any one of embodiments 7 to 10, wherein the linking group is indirectly linked via a linker to the moiety configured to support cell growth, viability, portability, or any combination thereof.

Embodiment 12. The microfluidic device of embodiment 11, wherein the linker comprises a triazolylene moiety.

Embodiment 13. The microfluidic device of any one of embodiments 1 to 12, wherein the at least one conditioned surface comprises saccharide moieties.

Embodiment 14. The microfluidic device of any one of embodiments 1 to 13, where the at least one conditioned surface comprises alkylene ether moieties.

Embodiment 15. The microfluidic device of any one of embodiments 1 to 14, wherein the at least one conditioned surface comprises amino acid moieties.

Embodiment 16. The microfluidic device of any one of embodiments 7 to 15, wherein the at least one conditioned surface comprises zwitterions.

Embodiment 17. The microfluidic device of any one of embodiments 1 to 16, wherein the conditioned surface comprises a cleavable moiety.

Embodiment 18. The microfluidic device of any one of embodiments 1 to 17, wherein the microfluidic device further comprises a substrate having a dielectrophoresis (DEP) configuration.

Embodiment 19. The microfluidic device of embodiment 18, wherein the DEP configuration is optically actuated.

Embodiment 20. The microfluidic device of any one of embodiments 1 to 19, wherein the at least one growth chamber comprises at least one surface conditioned to support cell growth, viability, portability, or any combination thereof of a mammalian cell.

Embodiment 21. The microfluidic device of any one of embodiments 1 to 20, wherein the at least one growth chamber comprises at least one surface conditioned to support cell growth, viability, portability, or any combination thereof of plant protoplast.

Embodiment 22. The microfluidic device of embodiment 21, wherein the plant protoplast is from an agricultural plant.

Embodiment 23. The microfluidic device of embodiment 22, wherein the plant protoplast is from a lettuce, tomato, corn, wheat, or tobacco plant.

Embodiment 24. The microfluidic device of any one of embodiments 1 to 23, wherein the at least one growth chamber comprises at least one surface conditioned to support cell growth, viability, portability, or any combination thereof a single plant cell and a corresponding clonal colony of plant cells.

Embodiment 25. A method of culturing at least one plant protoplast cell in a microfluidic device having a flow region configured to contain a flow of a first fluidic medium and at least one growth chamber, comprising the steps: introducing the at least one plant protoplast cell into the at least one growth chamber, wherein the at least one growth chamber is configured to have at least one surface conditioned to support cell growth, viability, portability, or any combination thereof, and, incubating the at least one plant protoplast cell for a period of time at least long enough to expand the at least one plant protoplast cell to produce a colony of plant protoplast cells.

Embodiment 26. The method of embodiment 25, wherein the microfluidic device is the microfluidic device of any one of embodiments 1 to 24.

Embodiment 27. The method of embodiment 25 or 26 further comprising: conditioning at least a surface of the at least one growth chamber.

Embodiment 28. The method of embodiment 27, wherein conditioning comprises treating the at least a surface of the at least one growth chamber with a conditioning reagent comprising a polymer.

Embodiment 29. The method of any one of embodiments 25 to 28, wherein introducing the at least one plant protoplast cell into the at least one growth chamber comprises using a dielectrophoresis (DEP) force having sufficient strength to move the at least one plant protoplast cell.

Embodiment 30. The method of embodiment 29, wherein the DEP force is optically actuated.

Embodiment 31. The method of any one of embodiments 25 to 30 further comprising: perfusing the first fluidic medium during the incubating step, wherein the first fluidic medium is introduced via at least one inlet port of the microfluidic device and exported via at least one outlet of the microfluidic device, wherein, upon export, the first fluidic medium optionally comprises components from the second fluidic medium.

Embodiment 32. The method of any one of embodiments 25 to 31 further comprising: cleaving one or more cleavable moieties of the conditioned surface after the incubating step, thereby facilitating export of the one or more plant protoplast cells out of the growth chamber or isolation region thereof and into the flow region.

Embodiment 33. The method of any one of embodiments 25 to 32 further comprising: exporting one or more plant protoplast cells out of the growth chamber or the isolation region thereof into the flow region.

Embodiment 34. The method of anyone of embodiments 25 to 33, wherein the protoplast is from a lettuce, tomato, corn, wheat, or tobacco plant.

Embodiment 35. The method of any one of embodiments 25 to 34, wherein introducing the at least one plant protoplast cell into the at least one growth chamber comprises introducing a single plant protoplast cell into the growth chamber, and wherein the colony of plant protoplast cells produced by the incubating step is a clonal colony.

Embodiment 36. The method of any one of embodiments 25 to 35, wherein the first fluidic medium is a growth medium that supports protoplast growth.

Embodiment 37. A method of identifying a plant protoplast that lacks pathogen resistance, the method comprising: introducing a first fluidic medium containing one or more protoplasts into a microfluidic device comprising an enclosure having a flow region and at least one growth chamber; moving a first protoplast of the one or more protoplasts into a first growth chamber of the at least one growth chamber; contacting the first protoplast with a pathogenic agent; and monitoring viability of the first protoplast during a first time period after contacting the first protoplast with the pathogenic agent, wherein protoplast viability at the end of the first time period indicates that the protoplast lacks resistance to the pathogenic agent.

Embodiment 38. The method of embodiment 37, wherein the one or more protoplasts are from a broad acre crop plant.

Embodiment 39. The method of embodiment 38, wherein the broad acre crop plant is a wheat, corn, soy, or cotton plant.

Embodiment 40. The method of embodiment 37, wherein the one or more protoplasts are from a high value or ornamental crop plant.

Embodiment 41. The method of embodiment 40, wherein the high value crop plant is a tomato, lettuce, pepper, or squash plant.

Embodiment 42. The method of embodiment 37, wherein the one or more protoplasts are from a turf or forage plant.

Embodiment 43. The method of embodiment 42, wherein the turf or forage plant is a grass or alfalfa plant.

Embodiment 44. The method of embodiment 37, wherein the one or more protoplasts are from an experimental plant (e.g., an Arabidopsis plant or an Antirrhinum plant).

Embodiment 45. The method of any one of embodiments 37 to 44, wherein the pathogenic agent is a plant pathogen or a molecule derived therefrom.

Embodiment 46. The method of embodiment 45, wherein the plant pathogen is a virus, a bacterium, or a fungal cell.

Embodiment 47. The method of embodiment 45 or 46, wherein the pathogenic agent is a molecular agent (e.g., a viral capsid protein, a flagellar protein, a lipopolysaccharide, a peptidoglycan, a chitin protein) or a fragment thereof.

Embodiment 48. The method of any one of embodiments 37 to 47, wherein contacting the first protoplast with the pathogenic agent comprises flowing a second fluidic medium containing the pathogenic agent into the flow region of the microfluidic device.

Embodiment 49. The method of embodiment 48, wherein contacting the first protoplast with the pathogenic agent further comprises moving the pathogenic agent into the isolation region of the first growth chamber or allowing the pathogenic agent to diffuse from the flow region into the isolation region of the first growth chamber.

Embodiment 50. The method of any one of embodiments 37 to 49, wherein said enclosure further comprises a base, a microfluidic circuit structure disposed on the base, and a cover.

Embodiment 51. The method of embodiment 50, wherein the cover and the base are part of a dielectrophoresis (DEP) mechanism for selective inducing DEP forces on micro-objects, and wherein moving the first protoplast into the first growth chamber comprises applying DEP force on the first protoplast.

Embodiment 52. The method of any one of embodiments 37 to 51, wherein the microfluidic device further comprises a first electrode, an electrode activation substrate, and a second electrode, wherein the first electrode is part of a first wall of the enclosure and the electrode activation substrate and the second electrode are part of a second wall of the enclosure, wherein the electrode activation substrate comprises a photoconductive material, semiconductor integrated circuits, or phototransistors, and wherein moving the first protoplast into the first growth chamber comprises applying DEP force on the first protoplast.

Embodiment 53. The method of embodiment 52, wherein the first wall is a cover, and wherein the second wall is a base.

Embodiment 54. The method of embodiment 52 or 53, wherein the electrode activation substrate comprises phototransistors.

Embodiment 55. The method of embodiment 50 or 53, wherein the cover and/or the base is transparent to light.

Embodiment 56. The method of any one of embodiments 37 to 55, wherein the first growth chamber is a sequestration pen that comprises an isolation region and a connection region that fluidically connects the isolation region to the flow region, and wherein the isolation region is an unswept region of the micro-fluidic device.

Embodiment 57. The method of embodiment 56, wherein the enclosure further comprises a microfluidic channel comprising at least a portion of the flow region, wherein the connection region of the sequestration pen comprises a proximal opening into the microfluidic channel having a width W_{con} ranging from about 50 microns to about 150 microns and a distal opening into the isolation region, and wherein a length L_{con} of the connection region from the proximal opening to the distal opening is as least 1.0 times the width W_{con} of the proximal opening of the connection region.

Embodiment 58. The method of embodiment 57, wherein the length L_{con} of the connection region from the proximal opening to the distal opening is at least 1.5 times the width W_{con} of the proximal opening of the connection region.

Embodiment 59. The method of embodiment 57, wherein the length L_{con} of the connection region from the proximal opening to the distal opening is at least 2.0 times the width W_{con} of the proximal opening of the connection region.

Embodiment 60. The method of any one of embodiments 57 to 59, wherein the width W_{con} of the proximal opening of the connection region ranges from about 50 microns to about 100 microns.

Embodiment 61. The method of any one of embodiments 57 to 60, wherein the length L_{con} of the connection region from the proximal opening to the distal opening is between about 50 microns and about 500 microns.

Embodiment 62. The method of any one of embodiments 57 to 61, wherein a height H_{ch} of the microfluidic channel at the proximal opening of the connection region is between 20 microns and 100 microns (e.g., between about 30 microns and 60 microns).

Embodiment 63. The method of any one of embodiments 57 to 62, wherein a width W_{ch} of the microfluidic channel at the proximal opening of the connection region is between about 50 microns and about 500 microns (e.g., between about 100 microns and 250 microns).

Embodiment 64. The method of any one of embodiments 56 to 63, wherein the volume of the isolation region of the sequestration pen ranges from about 5×10⁵ to about 5×10⁶ cubic microns.

Embodiment 65. The method of any one of embodiments 56 to 64, wherein the volume of the isolation region of the sequestration pen ranges from about 1×10⁶ to about 2×10⁶ cubic microns.

Embodiment 66. The method of any one of embodiments 56 to 65, wherein the proximal opening of the connection region is parallel to a direction of bulk flow in the flow region.

Embodiment 67. The method of any one of embodiments 37 to 66, wherein monitoring viability of the first protoplast during the first time period comprises monitoring cell division of the first protoplast, and wherein cell division of the first protoplast indicates that the protoplast lacks resistance to the pathogenic agent.

Embodiment 68. The method of any one of embodiments 37 to 67, wherein monitoring viability of the first protoplast during the first time period comprises maintaining the microfluidic chip at a temperature of about 20° C. to about 30° C. (e.g., about 24° C. to about 26° C.) during the first time period and/or minimizing the amount of light to which the first protoplast is exposed during the first time period (e.g., by maintaining the microfluidic chip in a dark environment or substantially blocking light external to the instrument from entering into the sequestration pen).

Embodiment 69. The method of any one of embodiments 37 to 68, wherein monitoring viability of the first protoplast during the first time period comprises periodically perfusing protoplast growth medium through the flow region of the microfluidic device during the first time period.

Embodiment 70. The method of embodiment 69, wherein the protoplast growth medium is perfused through the flow region no more than once per day (e.g., no more than once every two, three, four, five, six, seven, or more days).

Embodiment 71. The method of any one of embodiments 37 to 70, wherein monitoring viability of the first protoplast during the first time period comprises staining the first protoplast with a cell viability dye (e.g., fluorescein diacetate (i.e., FDA) or Hoechst).

Embodiment 72. The method of any one of embodiments 37 to 71, wherein monitoring viability of the first protoplast during the first time period comprises staining the first protoplast with a chlorophyll stain and/or a cell wall stain (e.g., calcofluor white).

Embodiment 73. The method of any one of embodiments 37 to 72, wherein the first time period is at least 12 hours.

Embodiment 74. The method of embodiment 74, wherein the first time period is at least 24, 48, 72, 96, 120 hours, or more (e.g., 7 days, 8 days, 9 days, 10 days, 11 days, 12 days, 13 days, 14 days, 15 days, 16 days, 17 days, 18 days, 19 days, 20 days, 21 days, or longer).

Embodiment 75. The method of any one of embodiments 37 to 74 further comprising: determining that the first protoplast lacks resistance to the pathogenic agent; and exporting the first protoplast from the first growth chamber and the microfluidic device.

Embodiment 76. The method of any one of embodiments 37 to 75 further comprising: determining that the first protoplast lacks resistance to the pathogenic agent; and sequencing one or more disease resistance genes of the first protoplast.

Embodiment 77. The method of any one of embodiments 37 to 76 further comprising: determining that the first protoplast lacks resistance to the pathogenic agent; and sequencing the transcriptome of the first protoplast.

Embodiment 78. The method of any one of embodiments 37 to 77 further comprising: determining that the first protoplast lacks resistance to the pathogenic agent; and sequencing the genome of the first protoplast.

Embodiment 79. The method of any one of embodiments 76 to 78 further comprising: identifying a molecular change or defect in the sequence of one or more disease resistance genes, the transcriptome, and/or the genome associated with the lack of pathogen resistance.

Embodiment 80. The method of any one of embodiments 37 to 79, the method further comprising: moving at least one protoplast into each of a plurality of growth chambers in the microfluidic device; and performing the remaining steps of the method on each of the protoplasts moved into the plurality of growth chambers.

Embodiment 81. A kit for screening a plant protoplast for a disease resistance trait, the kit comprising: a microfluidic chip, wherein the microfluidic chip comprises an enclosure having a flow region and at least one growth chamber; and a reagent for detecting viability of the plant protoplast.

Embodiment 82. The kit of embodiment 81 further comprising a surface conditioning reagent.

Embodiment 83. The kit of embodiment 81 or 82 further comprising a conditioning modification reagent, and wherein at least one surface of the growth chamber comprises a surface modifying ligand.

Embodiment 84. The kit of embodiment 81 or 82, wherein at least one surface of the growth chamber comprises a covalently linked coating material.

Embodiment 85. The kit of any one of embodiments 81 to 84, wherein the reagent for detecting the viability of the plant protoplast is a fluorescent stain (e.g., fluorescein diacetate (FDA), Hoechst, calcofluor white, a chlorophyll stain, or the like).

What is claimed:

1. A method of identifying a plant protoplast that lacks pathogen resistance, the method comprising:
introducing a first fluidic medium containing one or more protoplasts into a microfluidic device comprising an enclosure having a flow region and at least one growth chamber;
moving a first protoplast of the one or more protoplasts into a first growth chamber of the at least one growth chamber; wherein the first growth chamber is a sequestration pen that comprises an isolation region and a connection region that fluidically connects the isolation region to the flow region, and wherein the isolation region is an unswept region of the micro-fluidic device;
contacting the first protoplast with a pathogenic agent; and
monitoring viability of the first protoplast during a first time period after contacting the first protoplast with the pathogenic agent,
wherein protoplast viability at the end of the first time period indicates that the protoplast lacks resistance to the pathogenic agent.

2. The method of claim 1, wherein the one or more protoplasts are from a broad acre crop plant, a high value or ornamental crop plant, a turf or forage plant, or an experimental plant.

3. The method of claim 2, wherein the one or more protoplasts are from a broad acre crop plant, and the broad acre crop plant is a wheat, corn, soy, or cotton plant.

4. (canceled)

5. The method of claim 2, wherein the one or more protoplasts are from a high value or ornamental crop plant, and the high value or ornamental crop plant is a tomato, lettuce, pepper, or squash plant.

6. (canceled)

7. The method of claim 2, wherein the one or more protoplasts are from a turf or forage plant, and the turf or forage plant is a grass or alfalfa plant.

8. (canceled)

9. The method of claim 1, wherein the pathogenic agent is a plant pathogen or a molecule derived therefrom.

10. (canceled)

11. (canceled)

12. The method of claim 1, wherein
contacting the first protoplast with the pathogenic agent comprises flowing a second fluidic medium containing the pathogenic agent into the flow region of the microfluidic device.

13. The method of claim 12, wherein contacting the first protoplast with the pathogenic agent further comprises moving the pathogenic agent into the isolation region of the first growth chamber or allowing the pathogenic agent to diffuse from the flow region into the isolation region of the first growth chamber.

14. The method of claim 1, wherein said enclosure further comprises a base, a microfluidic circuit structure disposed on the base, and a cover.

15. The method of claim 14, wherein the cover and the base are part of a dielectrophoresis (DEP) mechanism for selective inducing DEP forces on micro-objects, and wherein moving the first protoplast into the first growth chamber comprises applying DEP force on the first protoplast.

16. The method of claim 1, wherein the microfluidic device further comprises a first electrode, an electrode activation substrate, and a second electrode, wherein the first electrode is part of a first wall of the enclosure and the electrode activation substrate and the second electrode are part of a second wall of the enclosure, wherein the electrode activation substrate comprises a photoconductive material, semiconductor integrated circuits, or phototransistors, and wherein moving the first protoplast into the first growth chamber comprises applying DEP force on the first protoplast.

17. (canceled)

18. (canceled)

19. (canceled)

20. (canceled)

21. The method of claim 1, wherein the enclosure further comprises a microfluidic channel comprising at least a portion of the flow region, wherein the connection region of the sequestration pen comprises a proximal opening into the microfluidic channel having a width W_{con} ranging from about 50 microns to about 150 microns and a distal opening into the isolation region, and wherein a length L_{con} of the connection region from the proximal opening to the distal opening is as least 1.0 times the width W_{con} of the proximal opening of the connection region.

22. The method of claim 21, wherein the length L_{con} of the connection region from the proximal opening to the distal opening is at least 1.5 times the width W_{con} of the proximal opening of the connection region or wherein the length L_{con} of the connection region from the proximal opening to the distal opening is at least 2.0 times the width W_{con} of the proximal opening of the connection region.

23. (canceled)

24. The method of claim 21, wherein the microfluidic device further comprises at least one of:
the width W_{con} of the proximal opening of the connection region ranges from about 50 microns to about 100 microns;
the length L_{con} of the connection region from the proximal opening to the distal opening is between about 50 microns and about 500 microns;
a height H_{ch} of the microfluidic channel at the proximal opening of the connection region is between 20 microns and 100 microns; and
a width W_{ch} of the microfluidic channel at the proximal opening of the connection region ranging between about 50 microns and about 500 microns.

25. (canceled)

26. (canceled)

27. (canceled)

28. The method of claim 20, wherein the volume of the isolation region of the sequestration pen ranges from about 5×10⁵ to about 5×10⁶ cubic microns or from about 1×10⁶ to about 2×10⁶ cubic microns.

29. (canceled)

30. The method of claim 20, wherein the proximal opening of the connection region is parallel to a direction of bulk flow in the flow region.

31. The method of claim 1, wherein monitoring viability of the first protoplast during the first time period comprises monitoring cell division of the first protoplast, and wherein cell division of the first protoplast indicates that the protoplast lacks resistance to the pathogenic agent.

32. The method of claim 1, wherein monitoring viability of the first protoplast during the first time period comprises at least one of:
maintaining the microfluidic chip at a temperature of about 20° C. to about 30° C. during the first time period;
minimizing the amount of light to which the first protoplast is exposed during the first time period; and
monitoring viability of the first protoplast during the first time period comprises periodically perfusing protoplast growth medium through the flow region of the microfluidic device during the first time period.

33. (canceled)

34. The method of claim 33, wherein the protoplast growth medium is perfused through the flow region no more than once every three days.

35. The method of claim 1, wherein monitoring viability of the first protoplast during the first time period comprises staining the first protoplast with at least one of a cell viability dye, chlorophyll stain, and cell wall stain.

36. (canceled)

37. The method of claim 1, wherein the first time period is at least 12 hours.

38. The method of claim 37, wherein the first time period is at least 96 hours.

39. The method of claim 1, further comprising:
determining that the first protoplast lacks resistance to the pathogenic agent; and
exporting the first protoplast from the first growth chamber and the microfluidic device.

40. The method of any claim 1, further comprising:
determining that the first protoplast lacks resistance to the pathogenic agent; and
sequencing at least one of one or more disease resistance genes, transcriptome, and/or a genome of the first protoplast.

41.-42. (canceled)

43. The method of claim 40 further comprising:
identifying a molecular change or defect in the sequence of one or more disease resistance genes, the transcriptome, and/or the genome associated with the lack of pathogen resistance.

44. The method of claim 1, the method further comprising:
moving at least one protoplast into each of a plurality of growth chambers in the microfluidic device; and
performing the remaining steps of the method on each of the protoplasts moved into the plurality of growth chambers.

45.-49. (canceled)
